(19) United States
(12) Patent Application Publication
(10) Pub. No.: US 2020/0179361 A1
(43) Pub. Date: Jun. 11, 2020

(54) COMPOSITIONS, METHODS OF USE, AND METHODS OF TREATMENT

(71) Applicant: TORREY PINES INSTITUTE FOR MOLECULAR STUDIES, PORT ST. LUCIE, FL (US)

(72) Inventors: Marcello GIULIANOTTI, Vero Beach, FL (US); Lawrence TOLL, Palm City, FL (US); Gregory S. WELMAKER, Palm City, FL (US); Jinhua WU, Palm City, FL (US); Yongping YU, Vero Beach, FL (US)

(21) Appl. No.: 16/614,668

(22) PCT Filed: May 21, 2018
(86) PCT No.: PCT/US2018/033666

§ 371 Date: Nov. 18, 2019

Related U.S. Application Data
(60) Provisional application No. 62/509,211, filed on May 22, 2017.

(51) Int. Cl. A61K 31/4439; A61P 25/34

(52) U.S. Cl. CPC A61K 31/4439; A61P 25/34

(57) ABSTRACT

The present disclosure provides compositions including a α4β2 nAChR antagonist, pharmaceutical compositions including a α4β2 nAChR antagonist, methods of making the compositions or pharmaceutical compositions, methods of treatment of a condition (e.g., nicotine addiction) or disease, methods of treatment using compositions or pharmaceutical compositions, and the like. Embodiments of the present disclosure can be used to reduce nicotine cravings and treat nicotine addiction. The compositions have selective affinity for the α4β2 receptor, which appears to be involved in nicotine dependence.

### CROSS-REFERENCE TO RELATED APPLICATION

This application claims the benefit of and priority to U.S. Provisional Patent Application No. 62/509,211, having the title “COMPOSITIONS, METHODS OF USE, AND METHODS OF TREATMENT,” filed on May 22, 2017, the entirety of which is hereby incorporated by reference.

### STATEMENT REGARDING FEDERALLY SPONSORED RESEARCH OR DEVELOPMENT

This invention was made with government support under R44DA036968 awarded by the National Institute on Drug Abuse within the National Institutes of Health. The government has certain rights in the invention.

### BACKGROUND

Worldwide, 6 million people—including 480,000 in the United States—die premature deaths from smoking-induced disease each year¹. Despite decades of public health efforts, 17.8% of U.S. adults continue to smoke, with direct medical costs averaging $170 billion/year and lost productivity costs averaging $156 billion/year^{1, 2}. Moreover, although 70% of smokers say they want to quit, smoking's powerful dependence results in at least a 90% failure^{1, 3}.

Nicotine addiction, like addiction to other psychomotor stimulants, is thought to be due to the activation of the dopaminergic mesocorticolimbic pathway^{8, 9}. The nicotinic receptor subtype most prevalent in this region is the α4β2 nAChR, although there is a significant level of the α6 subunit^{10, 11}. A great deal of evidence suggests that the α4β2 receptor is involved in nicotine dependence. In particular, 82-knockout mice do not self-administer nicotine^{8, 12}, the selective antagonists dihydro-β-erythroidine (DHβE) and 2-fluoro-3-(4-nitro-phenyl)deschloroepibatidine (4-nitro-PFEB) block nicotine self-administration^{13, 14}, and the α4β2 nAChR partial agonist varenicline is clinically used as a smoking cessation medication⁴. More recently, the importance of other nAChR subunits in nicotine self-administration has been demonstrated, particularly α6 and α5 in the ventra tegmental area (VTA), and α5 and β4 in the habenular-interpeduncular pathway¹⁵⁻¹⁸. Nevertheless, as demonstrated with varenicline, modulation of α4β2 nAChR clearly has promise as a mechanism to modulate smoking.

### SUMMARY

Embodiments of the present disclosure provide for compositions, methods of use, methods of treatment, methods of making the compounds, and the like.

An embodiment of the present disclosure, includes, among others, a composition comprising a α4β2 nAChR antagonist having the following structure:

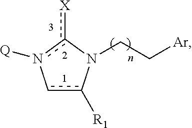

and stereoisomers thereof, wherein X is independently selected from H, NH₂, OH, an alkyl group, or a substituted alkyl group, wherein a double bond 3 is present based on X; R₁ is independently selected from an alkyl group, a substituted alkyl group, hydroxyl alkyl group, or a substituted hydroxyl alkyl group; Q is H or is not present and when not present, a double bond 2 is formed; n is 0, 1, 2, 3, 4 or 5; and Ar is independently selected from an heteroaryl group. In an aspect, H is optional present (on the two adjacent carbons of the ring (one carbon bonded to R₁)) and when not present double bond 1 can be present.

An embodiment of the present disclosure, includes, among others, a pharmaceutical composition, comprising a therapeutically effective amount of a α4β2 nAChR antagonist described above and herein or a pharmaceutically acceptable salt of the α4β2 nAChR antagonist, and a pharmaceutically acceptable carrier, to treat a condition. In an aspect, the α4β2 nAChR antagonist is described in the preceding paragraph.

An embodiment of the present disclosure, includes, among others, a method of treating a condition comprising: delivering to a subject in need thereof, a pharmaceutical composition as described above and herein, wherein the pharmaceutical composition includes a therapeutically effective amount of a α4β2 nAChR antagonist as described above or a pharmaceutically acceptable salt of the α4β2 nAChR antagonist, and a pharmaceutically acceptable carrier, to treat the condition. In an aspect, the α4β2 nAChR antagonist is described in the two paragraphs above this paragraph.

An embodiment of the present disclosure, includes, among others, a method of treatment for a condition (e.g., nicotine addiction, relapse of nicotine addiction, obsessive-compulsive disorder, depression, anxiety, panic disorder, anxiety, or generalized anxiety disorder) in a mammal of, the method comprising administering to a mammal in need thereof a pharmaceutically effective amount of a composition or pharmaceutical composition described above and herein, or a pharmaceutically acceptable salt thereof. In an aspect, the composition or pharmaceutical composition includes a α4β2 nAChR antagonist as described in the three paragraphs above this paragraph.

An aspect of the present disclosure includes method of making a compound, comprising the following reaction scheme:

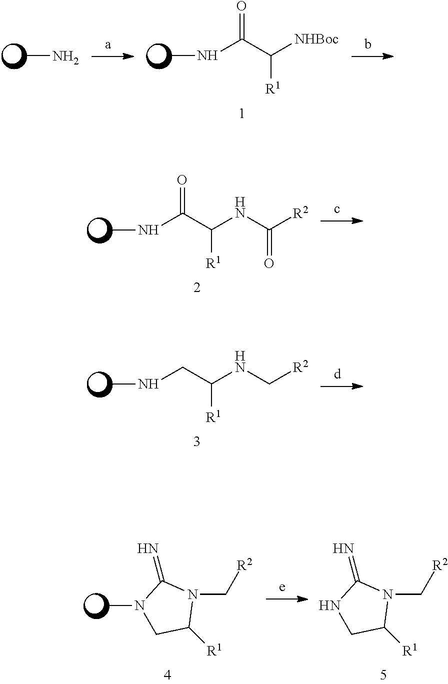

wherein a) comprises neutralizing p-methylbenzdrylamine and coupling Boc-amino acid to form compound 1; wherein b) comprises removing the Boc protecting group and coupling carboxylic acid using the Boc-amino acid to form compound 2; wherein c) comprises amide reduction to form compound 3; wherein d) comprises performing guanidine cyclization to form compound 4; and wherein e) comprises cleaving the resin (or protecting group) to form compound 5; and wherein R₁ is independently selected from an alkyl group, a substituted alkyl group, hydroxyl alkyl group, or a substituted hydroxyl alkyl group; and wherein R₂ is (CH₂)ₙ—, —Ar, n is 1 to 5, wherein Ar is independently selected from an heteroaryl group. In an aspect, the amine can be protected or include a resin.

An aspect of the present disclosure includes a method of making a compound, comprising the following reaction scheme:

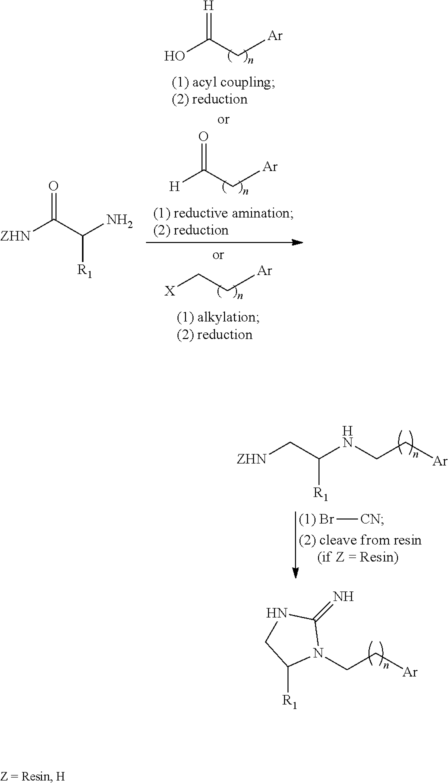

wherein an aminoamide, optionally bound to a resin (Z) or H, undergoes one of: acyl coupling or reductive amination or alkylation, to produce, after reduction of all amide groups, a diamine; wherein, the diamine is cyclized to the guanidine and then optionally the resin is cleaved; and wherein R₁ is independently selected from an alkyl group, a substituted alkyl group, hydroxyl alkyl group, or a substituted hydroxyl alkyl group; and wherein R₂ is —(CH₂)ₙ—Ar, n is 0 to 10 or 0 to 5, wherein Ar is independently selected from an heteroaryl group. In another aspect, Z can be a protecting group.

An aspect of the present disclosure includes a method of making a compound, comprising the following reaction scheme:

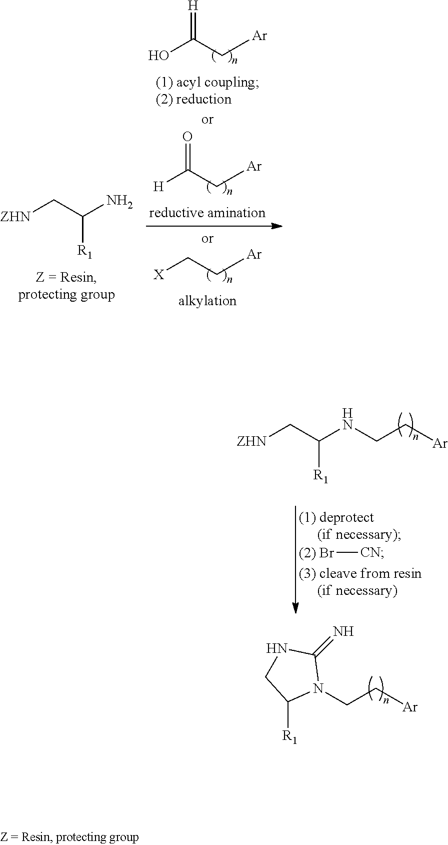

wherein a protected diamine undergoes one of (1) acyl coupling followed by reduction; or (2) reductive amination; or (3) alkylation, to provide the diamine compound; wherein, the diamine, if protected is deprotected and then cyclized to the guanidine or if resin bound is cyclized to the guanidine and then, optionally, the resin is cleaved; and wherein R₁ is independently selected from an alkyl group, a substituted alkyl group, hydroxyl alkyl group, or a substituted hydroxyl alkyl group; and wherein Ar is independently selected from an heteroaryl group and n is 0 to 10 or 0 to 5.

Other compositions, methods, features, and advantages will be or become apparent to one with skill in the art upon examination of the following drawings and detailed description. It is intended that all such additional compositions, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 shows Bis-cyclic guanidine scaffold contains compounds with moderate affinity for α4β2 nAChR.

FIG. 2 shows SAR of compounds synthesized.

FIG. 3 shows activity of AP-202 and TPI-211 on α4β2 nAChR in HEK cells. AP-202 and TPI-211 are devoid of agonist activity when tested alone, but potently block epibatidine-induced membrane potential change.

FIG. 4 shows inhibition of nicotine self-administration (*p<0.05 compared to vehicle control in a 120 min session (A) and nicotine reinstatement compared to vehicle control (1 h session) by 202. (B) Reinstatement was induced by nicotine prime (0.15 mg/kg s.c.) n=7 and (C) reinstatement was induced by cue.

FIG. 5 shows effects of (A) TPIMS-202 (B) TP 2212-59 on operant FR-3 nicotine and alcohol co-administration.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method may be carried out in the order of events recited or in any other order that is logically possible.

Embodiments of the present disclosure will employ, unless otherwise indicated, techniques of organic chemistry, biochemistry, molecular biology, pharmacology, medicine, and the like, which are within the skill of the art. Such techniques are explained fully in the literature.

Prior to describing the various embodiments, the following definitions are provided and should be used unless otherwise indicated.

### Definitions

Unless otherwise defined, all technical and scientific terms used herein have the same meaning as commonly understood by one of ordinary skill in the art of molecular biology, medicinal chemistry, and/or organic chemistry. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

The term “substituted” refers to any one or more hydrogens on the designated atom that can be replaced with a selection from the indicated group, provided that the designated atom's normal valence is not exceeded, and that the substitution results in a stable compound.

The term “aliphatic group” refers to a saturated or unsaturated linear or branched (substituted or unsubstituted) hydrocarbon group and encompasses alkyl, alkenyl, and alkynyl groups, for example.

As used herein, “alkyl” or “alkyl group” refers to a saturated aliphatic hydrocarbon radical which can be straight or branched (substituted or unsubstituted), having 1 to 20 carbon atoms, wherein the stated range of carbon atoms includes each intervening integer individually, as well as sub-ranges. Examples of alkyl include, but are not limited to methyl, ethyl, n-propyl, i-propyl, n-butyl, s-butyl, t-butyl, n-pentyl, and s-pentyl. The term “lower alkyl” means an alkyl group having less than 10 carbon atoms.

As used herein, “alkenyl” or “alkenyl group” refers to an aliphatic hydrocarbon radical which can be straight or branched (substituted or unsubstituted), containing at least one carbon-carbon double bond, having 2 to 20 carbon atoms, wherein the stated range of carbon atoms includes each intervening integer individually, as well as sub-ranges. Examples of alkenyl groups include, but are not limited to, ethenyl, propenyl, n-butenyl, i-butenyl, 3-methylbut-2-enyl, n-pentenyl, heptenyl, octenyl, decenyl, and the like.

The term “arylalkyl” refers to an arylalkyl group wherein the aryl and alkyl are as herein described (substituted or unsubstituted). Examples of arylalkyl include, but are not limited to, -phenylmethyl, phenylethyl, -phenylpropyl, -phenylbutyl, and -phenylpentyl.

The term “substituted,” as in “substituted alkyl”, “substituted cycloalkyl,” “substituted cycloalkenyl,” substituted aryl,” substituted biaryl,” “substituted fused aryl” as well as other substituted groups may contain in place of one or more hydrogens a group such as halogen, hydroxy, amino, halo, trifluoromethyl, cyano, —NH(lower alkyl), —N(lower alkyl)₂, lower alkoxy, lower alkylthio, or carboxy, and thus embraces the terms haloalkyl, alkoxy, fluorobenzyl, and the sulfur and phosphorous containing substitutions referred to below. In an embodiment, “substituted” includes the substituted group may contain in place of one or more hydrogens a group such as halogen or an alkyl group (e.g., a linear or branched C1 to C4 moiety).

As used herein, “halo”, “halogen”, or “halogen radical” refers to a fluorine, chlorine, bromine, and iodine, and radicals thereof. Further, when used in compound words, such as “haloalkyl” or “haloalkenyl”, “halo” refers to an alkyl or alkenyl radical in which one or more hydrogens are substituted by halogen radicals. Examples of haloalkyl include, but are not limited to, trifluoromethyl, trichloromethyl, pentafluoroethyl, and pentachloroethyl.

The term “alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. The term “lower alkoxy” means an alkoxy group having less than 10 carbon atoms.

The term “cycloalkyl” refers to a non-aromatic mono- or multicyclic ring system of about 3 to about 10 carbon atoms (substituted or unsubstituted), preferably of about 5 to about 10 carbon atoms. Preferred ring sizes of rings of the ring system include about 5 to about 6 ring atoms. Exemplary monocyclic cycloalkyl include cyclopentyl, cyclohexyl, cycloheptyl, and the like. Exemplary multicyclic cycloalkyl include 1-decalin, norbornyl, adamant-(1- or 2-)yl, and the like.

The term “cycloalkenyl” refers to a non-aromatic mono- or multicyclic ring system of about 3 to about 10 carbon atoms (substituted or unsubstituted), preferably of about 5 to about 10 carbon atoms, and which contains at least one carbon-carbon double bond. Preferred ring sizes of rings of the ring system include about 5 to about 6 ring atoms. Exemplary monocyclic cycloalkenyl include cyclopentenyl, cyclohexenyl, cycloheptenyl, and the like. An exemplary multicyclic cycloalkenyl is norbornylenyl.

The term “aryl” as used herein, refers to an aromatic monocyclic or multicyclic ring system of about 6 to about 14 carbon atoms (substituted or unsubstituted), preferably of about 6 to about 10 carbon atoms.

The term “heteroaryl” is used herein to denote an aromatic ring or fused ring structure of carbon atoms with one or more non-carbon atoms, such as oxygen, nitrogen, and sulfur, in the ring or in one or more of the rings in fused ring structures (substituted or unsubstituted). Examples are furanyl, pyranyl, thienyl, imidazyl, oxazolyl, pyrrolyl, pyridyl, pyrazolyl, pyrazinyl, pyrimidinyl, pyridineimidazoyl, indolyl, indazolyl, quinolyl, isoquinolyl, quinoxalyl, and quinazolinyl. Preferred examples are furanyl, indazolyl, imidazyl, oxazolyl, pyranyl, pyrrolyl, and pyridyl.

The term “biaryl” refers to an aryl, as defined above, where two aryl groups (substituted or unsubstituted) are joined by a direct bond or through an intervening alkyl group, preferably a lower alkyl group.

The term “fused aryl” refers to a multicyclic ring system (substituted or unsubstituted) as included in the term “aryl,” and includes aryl groups and heteroaryl groups that are condensed. Examples are naphthyl, anthryl and phenanthryl. The bonds can be attached to any of the rings.

“Aralkyl” and “heteroaralkyl” refer to aryl and heteroaryl moieties (substituted or unsubstituted), respectively, that are linked to a main structure by an intervening alkyl group, e.g., containing one or more methylene groups.

The term “unit dosage form,” as used herein, refers to physically discrete units suitable as unitary dosages for human and/or animal subjects, each unit containing a predetermined quantity of a compound (e.g., compositions or pharmaceutical compositions, as described herein) calculated in an amount sufficient to produce the desired effect in association with a pharmaceutically acceptable diluent, carrier or vehicle. The specifications for unit dosage forms depend on the particular compound employed, the route and frequency of administration, and the effect to be achieved, and the pharmacodynamics associated with each compound in the subject.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” as used in the specification and claims includes one and more such excipients, diluents, carriers, and adjuvants.

As used herein, a “pharmaceutical composition” is meant to encompass a composition or pharmaceutical composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

The term “therapeutically effective amount” as used herein refers to that amount of an embodiment of the composition or pharmaceutical composition being administered that will relieve to some extent one or more of the symptoms of the disease, i.e., nicotine addition, being treated, and/or that amount that will prevent, to some extent, one or more of the symptoms of the disease, i.e., nicotine addition, that the subject being treated has or is at risk of developing.

“Pharmaceutically acceptable salt” refers to those salts that retain the biological effectiveness and optionally other properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

In the event that embodiments of the disclosed compounds in the composition or pharmaceutical composition form salts, these salts are within the scope of the present disclosure. Reference to a compound used in the composition or pharmaceutical composition of any of the formulas herein is understood to include reference to salts thereof, unless otherwise indicated. The term “salt(s)”, as employed herein, denotes acidic and/or basic salts formed with inorganic and/or organic acids and bases. In addition, when a compound contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps which may be employed during preparation. Salts of the compounds of a compound may be formed, for example, by reacting the compound with an amount of acid or base, such as an equivalent amount, in a medium such as one in which the salt precipitates or in an aqueous medium followed by lyophilization.

Embodiments of the compounds of the composition or pharmaceutical composition of the present disclosure that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Embodiments of the compounds of the composition or pharmaceutical composition of the present disclosure that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the composition or pharmaceutical composition of the present disclosure are also contemplated herein.

To the extent that the disclosed the compounds of the composition or pharmaceutical composition of the present disclosure, and salts thereof, may exist in their tautomeric form, all such tautomeric forms are contemplated herein as part of the present disclosure.

All stereoisomers of the compounds of the composition or pharmaceutical composition of the present disclosure, such as those that may exist due to asymmetric carbons on the various substituents, including enantiomeric forms (which may exist even in the absence of asymmetric carbons) and diastereomeric forms are contemplated within the scope of this disclosure. Individual stereoisomers of the compounds of the disclosure may, for example, be substantially free of other isomers, or may be admixed, for example, as racemates or with all other, or other selected, stereoisomers. The stereogenic centers of the compounds of the present disclosure can have the S or R configuration as defined by the IUPAC 1974 Recommendations.

The term “prodrug” refers to an inactive precursor of the compounds of the composition or pharmaceutical composition of the present disclosure that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11, 345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

The term “administration” refers to introducing a composition of the present disclosure into a subject. One preferred route of administration of the composition is oral administration. Another preferred route is intravenous administration. However, any route of administration, such as topical, subcutaneous, peritoneal, intraarterial, inhalation, vaginal, rectal, nasal, introduction into the cerebrospinal fluid, or instillation into body compartments can be used.

As used herein, “treat”, “treatment”, “treating”, and the like refer to acting upon a condition, a disease or a disorder with a composition to affect the condition, disease or disorder by improving or altering it. The improvement or alteration may include an improvement in symptoms or an alteration in the physiologic pathways associated with the condition, disease, or disorder. “Treatment,” as used herein, covers one or more treatments of a disease (e.g., a condition (e.g., nicotine addiction, relapse of nicotine addiction, obsessive-compulsive disorder, depression, anxiety, panic disorder, anxiety, or generalized anxiety disorder), and related disorder or conditions) in a subject (e.g., a mammal, typically a human or non-human animal of veterinary interest), and includes: (a) reducing the risk of occurrence of the disease in a subject determined to be predisposed to the condition or disease but not yet diagnosed with it (b) impeding the development of the condition or disease, and/or (c) relieving the condition disease, e.g., causing regression of the condition or disease and/or relieving one or more disease symptoms.

As used herein, the terms “prophylactically treat” or “prophylactically treating” refers completely or partially preventing (e.g., about 50% or more, about 60% or more, about 70% or more, about 80% or more, about 90% or more, about 95% or more, or about 99% or more) a condition, a disease, or a symptom thereof and/or may be therapeutic in terms of a partial or complete cure for a condition, a disease, and/or adverse effect attributable to the disease.

As used herein, the term “subject,” or “patient,” includes humans and mammals (e.g., mice, rats, pigs, cats, dogs, and horses). Typical subjects to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject.

As used herein, “active agent” or “active ingredient” can refer to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “addiction” can be used to refer to a pathological (physical and/or mental) state, involving the progression of acute substance use to the development of substance-seeking behavior, the vulnerability to relapse, and the decreased, slowed ability to respond to naturally rewarding stimuli. The Diagnostic and Statistical Manual of Mental Disorders, Fourth Edition (DSM-IV) has categorized three stages of addiction: preoccupation/anticipation, bingelintoxication, and withdrawal/negative affect. These stages are characterized, respectively, everywhere by constant cravings and preoccupation with obtaining the substance; using more of the substance than necessary to experience the intoxicating effects; and experiencing tolerance, withdrawal symptoms, and decreased motivation for normal life activities. By the American Society of Addiction Medicine definition, substance addiction differs from substance dependence and substance tolerance. The term substance addiction is also used as a category which can include the same persons who can be given the diagnosis of substance dependence or substance abuse.

### Discussion

The present disclosure provides compositions including a α4β2 nAChR antagonist, pharmaceutical compositions including a α4β2 nAChR antagonist, methods of making the compositions or pharmaceutical compositions, methods of treatment of a condition (e.g., nicotine addiction) or disease, methods of treatment using compositions or pharmaceutical compositions, and the like. Embodiments of the present disclosure can be used to reduce nicotine cravings and treat nicotine addiction. The compositions have selective affinity for the α4β2 receptor, which appears to be involved in nicotine dependence. Additionally, the compositions of the present disclosure can also be used to attenuate co-morbid nicotine and alcohol cravings. Additional details are described in the Example.

An embodiment of the present disclosure includes a composition and pharmaceutical composition including a α4β2 nAChR antagonist. In an aspect, the pharmaceutical composition and the method of treatment of a disease or condition (e.g., of nicotine addiction and/or dependence, relapse of nicotine addiction, obsessive-compulsive disorder, depression, anxiety, panic disorder, anxiety, or generalized anxiety disorder) includes a therapeutically effective amount of a α4β2 nAChR antagonist, or a pharmaceutically acceptable salt of the α4β2 nAChR antagonist, and a pharmaceutically acceptable carrier, which can be optionally combined with another nAChR antagonist. An advantage of compositions of the present disclosure is that the anxiety-like behaviors induced by other treatments in the art may be avoided or reduced due the selectivity for α4β2 of the compounds in the present disclosure.

In an embodiment, the composition or pharmaceutical composition (as well as enantiomers thereof) includes a α4β2 nAChR antagonist that can be represented by the following general structure, as well as stereoisomers thereof, as well as pharmaceutically acceptable salts of these.

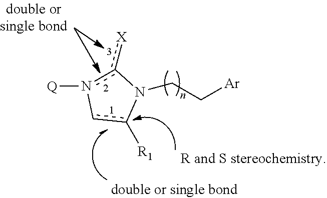

In an aspect, X can be selected from H, NH, NH₂, OH, an alkyl (e.g., C1 to C6 linear or branched alkyl group such as methyl, ethyl, propyl), or a substituted alkyl (e.g., substituted with a halogen such as F). R₁ can be selected from an alkyl (e.g., C1 to C6 linear or branched alkyl group such as methyl, ethyl, propyl), a substitute alkyl (e.g., substituted with a halogen such as F), a hydroxyl alkyl group, or a substituted hydroxyl alkyl group, where the carbon bonded to R₁ can have a R or S configuration. Q can be H or not present, and when not present, a double bond 2 is formed. Subscript “n” can be 0 to 10 or 0, 1, 2, 3, 4, or 5. Ar can be selected from a heteroaryl group (substituted or unsubstituted). A double bond 3 may be optionally present based on the selection of X (e.g., NH). A double bond 1 may be optionally present, and when not present, H is present to maintain a normal valence.

In an embodiment, X can be a methyl group or hydroxyl alkyl group and Ar can be selected from pyridyl, imidazoyl, pyridineimidazoyl, oxazolyl, or a halogen substituted pyridyl (each substituted or unsubstituted).

In an embodiment, the α4β2 nAChR antagonist can be selected from:

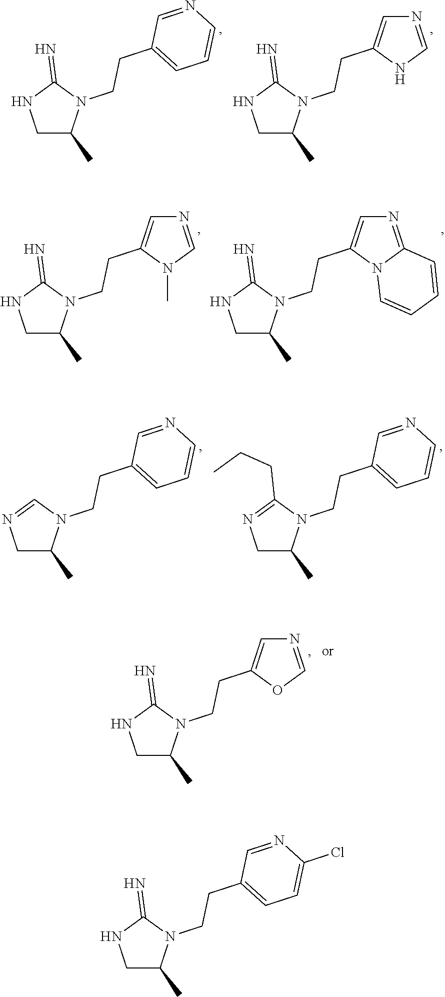

(substituted or unsubstituted), as well as stereoisomers of each.

In an embodiment, the α4β2 nAChR antagonist can be

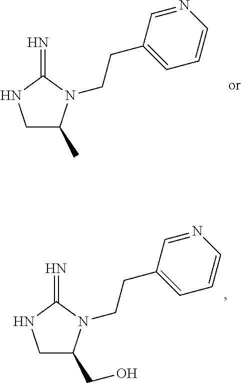 [SMILES: C[C@H]1CNC(=N)N1CCc1cccnc1 ; N=C1NC[C@H](CO)N1CCc1cccnc1]

(substituted or unsubstituted), as well as stereoisomers of each.

It should be noted that the therapeutically effective amount to result in uptake of the α4β2 nAChR antagonist(s) (e.g., each either alone or in combination with one another) into the subject will depend upon a variety of factors, including for example, the age, body weight, general health, sex, and diet of the subject; the time of administration; the route of administration; the rate of excretion of the specific compound employed; the duration of the treatment; the existence of other drugs used in combination or coincidental with the specific composition employed; and like factors well known in the medical arts.

### Pharmaceutical Formulations and Routes of Administration

Embodiments of the present disclosure include a α4β2 nAChR antagonist as identified herein and can be formulated with one or more pharmaceutically acceptable excipients, diluents, carriers and/or adjuvants. In addition, embodiments of the present disclosure include a α4β2 nAChR antagonist formulated with one or more pharmaceutically acceptable auxiliary substances. In particular α4β2 nAChR antagonist can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, the α4β2 nAChR antagonist can be administered to the subject using any means capable of resulting in the desired effect. Thus, the α4β2 nAChR antagonist can be incorporated into a variety of formulations for therapeutic administration. For example, the α4β2 nAChR antagonist can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the α4β2 nAChR antagonist may be administered in the form of its pharmaceutically acceptable salts, or a subject active composition may be used alone or in appropriate association, as well as in combination, with other pharmaceutically active compounds. The following methods and excipients are merely exemplary and are in no way limiting.

For oral preparations, the α4β2 nAChR antagonist can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the α4β2 nAChR antagonist can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the α4β2 nAChR antagonist can be utilized in aerosol formulation to be administered via inhalation. Embodiments of the α4β2 nAChR antagonist can be formulated into pressurized acceptable propellants such as dichlorodifluoromethane, propane, nitrogen and the like.

Furthermore, embodiments of the α4β2 nAChR antagonist can be made into suppositories by mixing with a variety of bases such as emulsifying bases or water-soluble bases. Embodiments of the α4β2 nAChR antagonist can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise the α4β2 nAChR antagonist in a composition as a solution in sterile water, normal saline or another pharmaceutically acceptable carrier.

Embodiments of the α4β2 nAChR antagonist can be formulated in an injectable composition in accordance with the disclosure. Typically, injectable compositions are prepared as liquid solutions or suspensions; solid forms suitable for solution in, or suspension in, liquid vehicles prior to injection may also be prepared. The preparation may also be emulsified or the active ingredient (triamino-pyridine derivative and/or the labeled triamino-pyridine derivative) encapsulated in liposome vehicles in accordance with the present disclosure.

In an embodiment, the α4β2 nAChR antagonist can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of the α4β2 nAChR antagonist can be accomplished using any of a variety of refillable, pump systems. Pumps provide consistent, controlled release over time. In some embodiments, the α4β2 nAChR antagonist can be in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, an active agent (e.g., the α4β2 nAChR antagonist) can be delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for the α4β2 nAChR antagonist are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the α4β2 nAChR antagonist adequate to achieve the desired state in the subject being treated.

Compositions of the present disclosure can include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) can be delivered in a controlled release system. For example, the α4β2 nAChR antagonist may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure (as well as combination compositions separately or together) include those formed by impregnation of the α4β2 nAChR antagonist described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### Dosages

Embodiments of the α4β2 nAChR antagonist can be administered to a subject in one or more doses. Those of skill will readily appreciate that dose levels can vary as a function of the specific the α4β2 nAChR antagonist administered, the severity of the symptoms and the susceptibility of the subject to side effects. Preferred dosages for a given compound are readily determinable by those of skill in the art by a variety of means.

In an embodiment, multiple doses of the α4β2 nAChR antagonist are administered. The frequency of administration of the α4β2 nAChR antagonist can vary depending on any of a variety of factors, e.g., severity of the symptoms, and the like. For example, in an embodiment, the α4β2 nAChR antagonist can be administered once per month, twice per month, three times per month, every other week (qow), once per week (qw), twice per week (biw), three times per week (tiw), four times per week, five times per week, six times per week, every other day (god), daily (qd), twice a day (qid), or three times a day (tid). As discussed above, in an embodiment, the α4β2 nAChR antagonist is administered continuously.

The duration of administration of the α4β2 nAChR antagonist analogue, e.g., the period of time over which the α4β2 nAChR antagonist is administered, can vary, depending on any of a variety of factors, e.g., patient response, etc. For example, the α4β2 nAChR antagonist in combination or separately, can be administered over a period of time of about one day to one week, about two weeks to four weeks, about one month to two months, about two months to four months, about four months to six months, about six months to eight months, about eight months to 1 year, about 1 year to 2 years, or about 2 years to 4 years, or more.

### Routes of Administration

Embodiments of the present disclosure provide methods and compositions for the administration of the active agent (e.g., the α4β2 nAChR antagonist) to a subject (e.g., a human) using any available method and route suitable for drug delivery, including in vivo and ex vivo methods, as well as systemic and localized routes of administration.

Routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. An active agent (e.g., the α4β2 nAChR antagonist) can be administered in a single dose or in multiple doses.

Embodiments of the α4β2 nAChR antagonist can be administered to a subject using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the α4β2 nAChR antagonist. Where systemic delivery is desired, administration typically involves invasive or systemically absorbed topical or mucosal administration of pharmaceutical preparations.

In an embodiment, the α4β2 nAChR antagonist can also be delivered to the subject by enteral administration. Enteral routes of administration include, but are not limited to, oral and rectal (e.g., using a suppository) delivery.

Methods of administration of the α4β2 nAChR antagonist through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### Example

Worldwide, 6 million people—including 480,000 in the United States—die premature deaths from smoking-induced disease each year¹. Despite decades of public health efforts, 17.8% of U.S. adults continue to smoke, with direct medical costs averaging $170 billion/year and lost productivity costs averaging $156 billion/year^{1, 2}. Moreover, although 70% of smokers say they want to quit, smoking's powerful dependence results in at least a 90% failure^{1, 3}.

Current FDA-approved pharmacotherapies to facilitate smoking cessation include nicotine replacement therapy (patches, gums, inhalers, and nasal sprays), the antidepressant bupropion, and varenicline. Unfortunately, cessation rates in most clinical trials of these therapies still average only from 10-20% for nicotine replacement therapy, from 15-25% for bupropion and from 23-40% for varenicline⁴⁻⁸. Although varenicline is the most successful treatment currently in clinical use, some users have become plagued by significant psychological problems including depression and thoughts of suicide⁷. Additional nicotine pharmacotherapies are in clinical trials, including the nonselective nicotinic acetylcholine receptor (nAChR) antagonist mecamylamine, the opioid antagonist naltrexone, and nicotine monoclonal antibody therapy. Clearly new medications are a high priority.

Nicotine addiction, like addiction to other psychomotor stimulants, is thought to be due to the activation of the dopaminergic mesocorticolimbic pathway^{8, 9}. The nicotinic receptor subtype most prevalent in this region is the α4β2 nAChR, although there is a significant level of the α6 subunit^{10, 11}. A great deal of evidence suggests that the α4β2 receptor is involved in nicotine dependence. In particular, β2-knockout mice do not self-administer nicotine^{8, 12}, the selective antagonists dihydro-β-erythroidine (DHβE) and 2-fluoro-3-(4-nitro-phenyl)deschloroepibatidine (4-nitro-PFEB) block nicotine self-administration^{13, 14}, and the α4β2 nAChR partial agonist varenicline is clinically used as a smoking cessation medication⁴. More recently, the importance of other nAChR subunits in nicotine self-administration has been demonstrated, particularly α6 and α5 in the ventra tegmental area (VTA), and α5 and β4 in the habenular-interpeduncular pathway¹⁵⁻¹⁸. Nevertheless, as demonstrated with varenicline, modulation of α4β2 nAChR clearly has promise as a mechanism to modulate smoking.

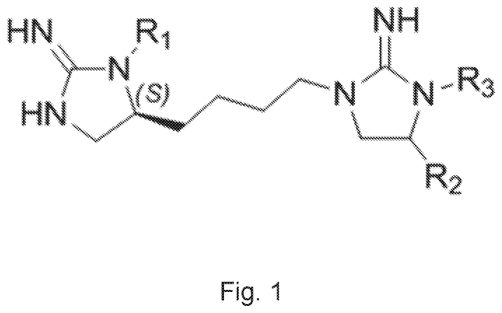

The Torrey Pines Institute for Molecular Studies (TPIMS) has a collection of small molecule libraries arranged in systematically formatted mixtures¹⁹. Computational analysis of the TPIMS small molecule libraries demonstrates that the collection covers novel areas of chemical space as well as structural features not available in other compound collections²⁰. The TPIMS collection contains over 5 million individual small molecules; however, due to the formatting of the mixtures, one can identify active individual compounds with moderate throughput capabilities (typically, fewer than 300-500 samples need to be tested). Utilizing the TPIMS small molecule library collection, compounds were screened for binding affinity at α4β2 nAChR and α3β4 nAChR. Initially, a “scaffold-ranking library” was tested. This library was made up of 37 mixtures, each mixture containing on average 135,000 structurally analogous compounds (identical scaffolds). One mixture, the one containing the bis-cyclic guanidines (FIG. 1), showed significant inhibition of [³H]epibatidine binding. After screening the positional scanning library associated with this mixture, we identified a series of individual analogs of these compounds, some of which demonstrated low micromolar (μM) affinity at α4β2 nAChR. Several of these analogs show >10 fold selectivity at α4β2 over α3β4 nAChR. All of these steps are discussed in detail in our recent publication²¹. From the compounds identified by library screening, traditional medicinal chemistry was initiated. Herein we discuss high affinity compounds that were identified as selective α4β2 nAChR pure antagonists and provide details of one compound in particular [5 (AP-202)] that showed significant activity in decreasing nicotine taking and seeking behaviors.

### Results

### SAR Studies.

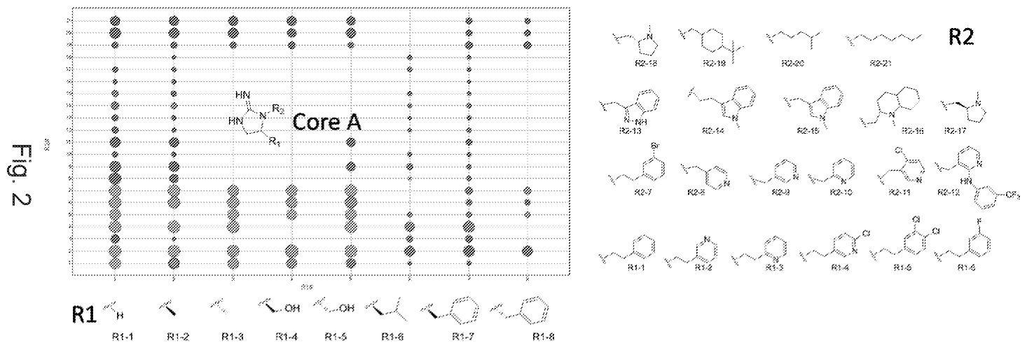

Utilizing 16 R1 and 21 R2 functionalities, over 140 different analogs with Core A were made (FIG. 2, note not all combinations of R groups were made). The compounds were initially tested for ability to inhibit [³H]epibatidine binding at a single concentration (10 μM) at both α4β2 and α3β4 nAChR and representative data is shown in FIG. 2 [8 R1 groups (x-axis) and 21 R2 groups (y-axis)]. Each dot in FIG. 2 represents a compound tested. The dots are color coded by α3β4 affinity [using a color gradient: red (0%, no binding) to green (100%, binding affinity)] and sized by α4β2 affinity (larger is higher affinity). A larger green dot binds to both targets at 10 μM, a large red dot indicates binding only to α4β2 at 10 μM and a small green dot indicates binding only to α3β4 at 10 μM. Many of the compounds that do not exhibit α4β2 selectivity at 10 μM (large green dots) are selective for α4β2 upon dose response. R group functionalities play a critical role in both affinity and selectivity. Increasing size at the R1 position significantly reduces the affinity of the compound (this trend was seen for an additional 8 R1 groups tested, data not shown). For the R2 position, specific substituted aromatic functionalities provide the highest affinity compounds. Also, attachment position to the pyridyl ring (i.e. R2-2 to R2-3) or the length of the carbon chain (i.e. R2-2 to R2-9) had a significant effect on affinity. Of note, some of the compounds containing an aliphatic group at the R2 position maintained α4β2 affinity, albeit at reduced levels, while exhibiting no α3β4 affinity at 10 μM (R2-20).

Based on this data, compounds were selected for Ki determination. Table 1 contains the data reported in nanomole (nM) from a series of compounds in which 2-(pyridine-3-yl)ethyl (R2: 2 from FIG. 2) is fixed in the R2 position. The compounds in this series all show selectivity toward α4β2 with a shift in affinity favoring the small R1 substitutions (hydrogen, methyl, and hydroxymethyl) as previously noted in FIG. 2. We next determined the Ki values for a series of analogs where the R1 position was fixed with one of the small R1 functionalities and R2 was adjusted (Table 2). Using either 2-(pyridine-3-yl)ethyl or 2-(6-chloropyridin-3-yl)ethyl at the R2 position produced potent and α4β2 nAChR-selective compounds (24.11 nM with a 11-fold selectivity and 13.13 nM with a 74-fold selectivity). Utilization of a non-pyridyl aromatic group at R2 either eliminates or slightly inverts the desired selectivity (i.e. compounds 1, 3, 17, and 21) and shortening the R2 linker carbon chain significantly reduces the affinity against both targets (15 vs 8 and 14 vs 5). Compounds with highest affinity and selectivity for α4β2 over α3β4 nAChR were 5 and 13.

5 and 13, were tested for binding affinity to α4β2, α3β4, α4β4, α3β2, and α3β4α5 nAChR in membranes from cells transfected with these receptors. As seen in Table 3, 5 and 13 have higher affinity for β2-containing nAChR over β4-containing receptor, with 14-fold or greater selectivity for α4β2 over α3β4, α4β4 and α3β4α5 nAChR.

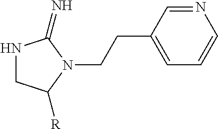

TABLE 1
Binding affinity and selectivity over α4β2 and α3β4 nAChR of a series of compounds
where 2-(pyridine-3-yl)ethyl (R2: 2 from Figure 2) is fixed in the R2 position

| # | R | α3β4 | α4β2 | Selectivity |
|---|---|---|---|---|
| 4 | S-benzyl | 12348 ± 1861 | 771 ± 334 | 16 |
| 5 | S-methyl | 1026 ± 254 | 18 ± 3 | 57 |
| 6 | S-isobutyl | 18683 ± 2058 | 4964 ± 03 | 4 |
| 7 | S-2-butyl | 18107 ± 529 | 863 ± 3 | 21 |
| 8 | hydrogen | 644 ± 17 | 58 ± 3 | 11 |
| 9 | S-isopropryl | 24487 ± 622 | 577 ± 25 | 42 |
| 10 | S-3-propylguanidine | 24043 ± 1073 | 1531 ± 30 | 16 |
| 11 | S-4-hydroxylbenzyl | 27420 ± 4396 | 1606 ± 41 | 17 |
| 12 | S-2-(methylthio)ethyl | 35590 ± 73 | 448 ± 14 | 79 |
| 13 | R-hydroxymethyl | 964 ± 93 | 13 ± 1 | 74 |

*All experiment are performed in duplicate or triplicate and repeated for 2 to 3 times.
Data are presented as Mean ± SEM.

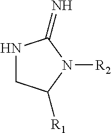

TABLE 2
Compounds binding affinity and selectivity over α4β2 and α3β4 nAChR of a series
of compounds having three small R1 functionalities with an adjusted R2 group

| # | R1 | R2 | α3β4 | α4β2 | Selectivity |
|---|---|---|---|---|---|
| 1 | hydrogen | 2-(3,4-dichlorophenyl)ethyl | 711 ± 18 | 1296 ± 44 | 0.5 |
| 2 | hydrogen | pyridine-4-ylmethyl | 50107 ± 1806 | 652 ± 166 | 77 |
| 3 | hydrogen | phenylethyl | 325 ± 40 | 361 ± 1 | 0.9 |
| 5 | S-methyl | 2-(pyridine-3-yl)ethyl | 1026 ± 254 | 18 ± 3 | 57 |
| 8 | hydrogen | 2-(pyridine-3-yl)ethyl | 644 ± 17 | 58 ± 3 | 11 |
| 13 | R-hydroxymethyl | 2-(pyridine-3-yl)ethyl | 964 ± 193 | 13 ± 1 | 74 |
| 14 | S-methyl | pyridine-3-ylmethyl | 36547 ± 729 | 1358 ± 9 | 27 |
| 15 | hydrogen | pyridine-3-ylmethyl | 98927 ± 1106 | 3750 ± 81 | 26 |
| 16 | S-methyl | phenylethyl | N/A | 2250 ± 20 | N/A |
| 17 | R-hydroxymethil | 2-(3,4-dichlorophenyl)ethyl | 321 ± 33 | 1045 ± 91 | 0.3 |
| 18 | hydrogen | 3-fluorophenylethyl | 633 ± 6 | 221 ± 17 | 3 |
| 19 | S-methyl | 3-fluorophenylethyl | N/A | 474 ± 12 | N/A |
| 20 | R-hydroxymethyl | 3-fluorophenylethyl | N/A | 704 ± 36 | N/A |
| 21 | hydrogen | 3-bromophenylethyl | 314 ± 26 | 601 ± 42 | 0.5 |
| 22 | S-methyl | 3-bromophenylethyl | 628 ± 61 | 361 ± 30 | 1.7 |
| 23 | R-hydroxymethyl | 3-bromophenylethyl | 1090 ± 123 | 640 ± 72 | 1.7 |
| 24 | S-methyl | 2-(6-chloropyridin-3-yl)ethyl | 125 ± 6 | 11 ± 1 | 11 |
| 25 | hydrohen | 2-(6-chloropyridin-3-yl)ethyl | 471 ± 16 | 80 ± 5 | 6 |
| 26 | R-hydroxymethyl | 2-(6-chloropyridin-3-yl)ethyl | 281 ± 19 | 20 ± 2 | 14 |

*All experiments are performed in duplicate or triplicate and repeated for 2 to 3 times.
Data are presented as Mean ± SEM.

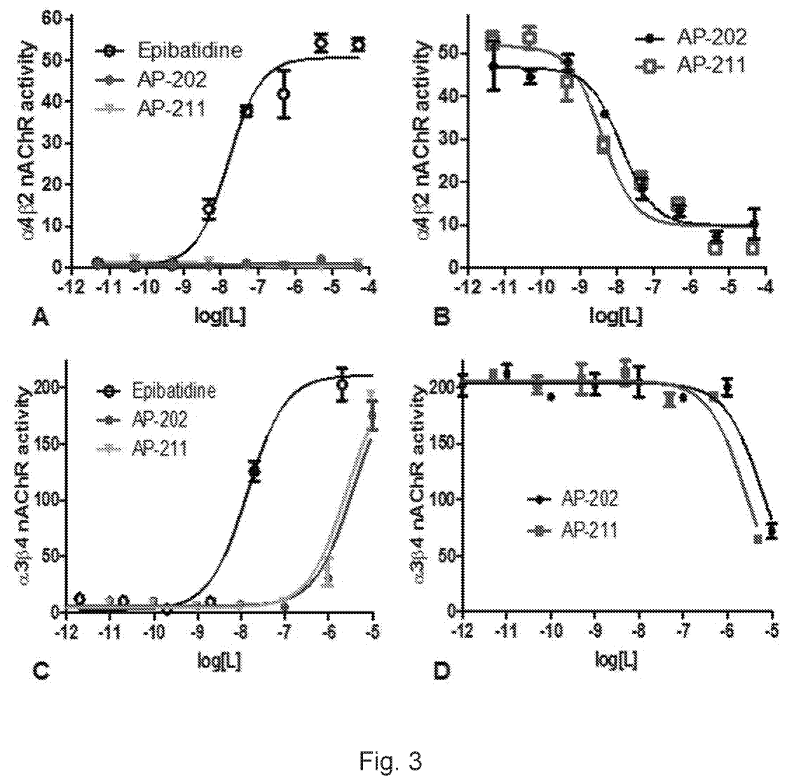

FIG. 3 shows the in vitro activity for 5 and 13. Compounds were tested for their ability to induce a change in membrane potential or Ca²⁺ flux in HEK cells transfected with rat α4β2 nAChR and α3β4 nAChR respectively. Both 5 and 13 (AP-211) are devoid of agonist activity in this in vitro model (FIG. 3A) and both are very potent inhibitors of epibatidine-induced changes in membrane potential in cells containing α4β2 nAChR, with IC₅₀s of approximately 10 nM (FIG. 3B). In cells containing α3β4 nAChR, these compounds have weak agonist activity with EC₅₀ values of 3509 nM and 2538 nM (FIG. 3C) and also weakly desensitize the receptor with IC₅₀ values of 6730 nM and 2717 nM respectively (FIG. 3D). Neither of these compounds have ability to activate α7 nAChR or block acetylcholine induced changes in membrane potential in cells transfected with α7 nAChR (data not shown). Therefore, these compounds are high affinity and selective α4β2 nAChR antagonists.

Our lead compounds 5 and 13 possess many properties that are in the desirable range for orally-available central nervous system (CNS)-active drugs and are compared to the mean values for marketed CNS drugs²² shown in Table 4.

TABLE 3
Ki (nM) values for 5 and 13 at various nAChR subtypes

| Com- | | | | | |
|---|---|---|---|---|---|
| pound | α4β2 | α3β4 | α4β4 | α3β2 | α3β4α5 |
| 5 | 18.0 ± 2.68 | 1026 ± 254 | 716 ± 200 | 167 ± 20.4 | 1619 ± 93.7 |
| 13 | 13.06 ± 0.55 | 964 ± 193 | 184 ± 28.7 | 85.4 ± 9.64 | 957 ± 70.6 |

*All experiments are performed in triplicate and repeated for 3 to 4 times.
Data are presented as Mean ± SEM

TABLE 4

| | | | Mean Value for |
|---|---|---|---|
| | | | Marketed CNS |
| Compound Property | 202 | 211 | Drugs |
| Mol Weight (MW) | 204 | 220 | 310 |
| Total # of O and N | 4 | 5 | 4.3 |
| ClogP | 0.2 | −0.8 | 2.5 |
| tPSA | 51 | 72 | 60-70 (<90) |

### In Vivo Activity

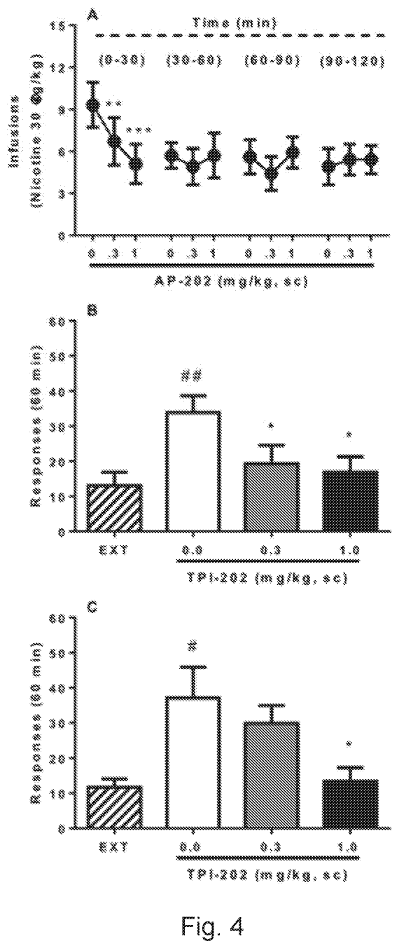

5 was tested for its ability to block nicotine self-administration in 2-hour operant sessions. Response rate at the end of the experiment was 25.4±4.4 infusions for the vehicle group, 21.4±4.4 for the 5, 0.3 mg/kg group and 22.1±4.8 for the 5, 1.0 mg/kg group, respectively. Initial one-way analysis of variance (ANOVA) conducted on the cumulative infusions obtained in 2-h sessions revealed no effect of 5 treatment (F_{(2,12)}=2.7, NS) (FIG. 4A). However, by analyzing the infusions obtained in 30-min intervals the experiment led to different results with 5 showing effectiveness in significantly attenuating nicotine self-administration. In fact, two-way ANOVA revealed significant interaction “time point×treatment” (F_{(6,36)}=4.1, p<0.01). Post hoc analysis indicated that 5 treatment decreased nicotine self-administration during the first 30 min but not during the remaining 90-min period of the session, thus suggesting short-term activity of the antagonist (FIG. 4B). Additionally, both doses examined of 5, 0.3 and 1.0 mg/kg, were effective (p<0.01, p<0.001, respectively). These data are consistent with other α4β2 nAChR antagonists, which also block nicotine self-administration^{13, 14}.

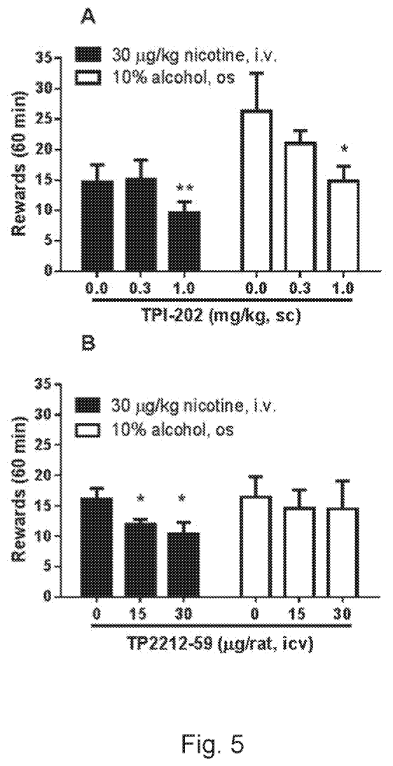

Subsequently, 5 was tested for its ability to reduce nicotine-seeking behavior. As shown in FIG. 5A, ANOVA analysis performed on the nicotine-associated lever indicated a main effect of 0.15 mg/kg nicotine priming in inducing nicotine-seeking behavior, as compared with extinction conditions (F_{(1,6)}=19.5, p<0.01). In examining the effect of pretreatment of 5 before the priming injection of nicotine, the overall ANOVA indicated a significant effect that 5 decreased responding to a nicotine priming (F_{(2,12)}=3.9, p<0.05). Post hoc comparisons revealed that both doses of 0.3 and 1.0 mg/kg significantly decreased responding (p<0.05 for both doses).

Conditioned stimuli previously associated to nicotine infusions were also able to elicit reinstatement of nicotine seeking (F_{(1,6)}=7.9, p<0.05). 5 could attenuate this effect [F_{(2,6)}=5.1, p<0.05]. Post hoc comparison tests revealed that 5 at the dose of 1 mg/kg resulted in a diminished number of responses as compared with those of the vehicle group (p<0.05, FIG. 5B).

5 was tested to determine whether it could block nicotine-induced hypothermia, an effect that can be mimicked by other selective and non-selective nAChR agonists²³. Overall treatments significantly modified body temperature. ANOVA showed “treatment”×“time point” interaction (F_{(12,84)}=7.3, p<0.001) with pairwise comparisons showing reduced rat core body temperature due to subcutaneous (sc) administration of 0.5 mg/kg of nicotine (p<0.001 at 15- and 30-min time point). However, 5 (1.0 mg/kg, sc) was neither able to induce hypothermia in rats nor to block nicotine-induced hypothermia (FIG. 6). These data suggest that nAChR subunits other than α4β2 could be implicated in mediating thermoregulatory effects of nicotine.

To determine the pharmacokinetic parameters of 5, relative blood content and blood brain barrier penetration were examined in rats following sc administration of 5 (2.0 mg/kg). Results indicate that 5 is taken up rapidly into the blood (F_{(3,9)}=17.7, p<0.001) and brain (F_{(3,9)}=8.7, p<0.01), reaching maximal concentration within 10 min, with a half-life of less than 1 h (blood: T=10, p<0.001; T=30, p<0.05; brain: T=10, p<0.01; T=30, p<0.01; FIG. 7). During this time, the brain concentration of 5 approached that of the blood concentration. This data is consistent with the short acting effect that we have demonstrated on nicotine self-administration.

### Discussion

Agents active at the nAChR have been actively studied for their ability to attenuate both nicotine and alcohol use in both preclinical and clinical settings. The most successful compound has been varenicline. This compound, which has had several billion dollars in sales as Chantix, is a potent α4β2 nAChR partial agonist. Although very potent and selective in binding studies (1000 fold selective for α4β2 over α3β4 or α7 nAChR), in vitro efficacy studies have determined that it is in fact a relatively low efficacy partial agonist at α4β2 nAChR, with EC₅₀ in the 1 μM range. Surprisingly, varenicline has somewhat similar potency and full agonist activity at α3β4 and α7 nAChR²⁴. However, varenicline potently desensitizes the receptor and thereby acts as a functional antagonist at nanomolar concentrations in vitro^{25, 26}. It may be desensitization combined with partial agonist activity^{27, 28}, that leads to the full complement of varenicline's activities in vivo. Other investigators have suggested conversely that the α3β4 or α7 activity might be the component that reduces nicotine consumption^{24, 25}. In fact varenicline blocks self-administration of both nicotine and alcohol, and it has been suggested that the α3β4 nAChR activity mediates the reduction in consumption of both drugs^{25, 29}. Our previous work suggests otherwise, as we have demonstrated that the selective α3β4 nAChR partial agonist AT-1001 can block nicotine self-administration at doses that do not affect alcohol self-administration^{26, 30} Furthermore, activation of α4-containing nAChRs has been shown to be necessary and sufficient for varenicline to reduce alcohol consumption³¹. Varenicline as a smoking cessation medication has also been plagued by reports of neuropsychiatric side effects^{7, 32}. Although the black box warning has been removed by the FDA, labeling still states postmarketing studies have reported serious or clinically significant neuropsychiatric adverse events in patients treated with the drug. Finally, although better than placebo and other medications in double blind studies, a recent open label study for smoking cessation has found varenicline to be equivalent to nicotine replacement therapy at six month and year time points³³. Ultimately it is not clear whether the side effects and moderate efficacy are due to the fact that varenicline has partial agonist activity at α4β2 nAChR, or that it has agonist activity at other nAChR such as α3β4 and α7 nAChR.

Many additional nAChR active agents have been tested in animal models and clinical trials. Cytisine, a nAChR partial agonist and a close analog of varenicline, is self-administered in mice³⁴ and has shown some efficacy in a limited number of clinical trials for smoking cessation³⁸. The partial agonist sazetidine-A, can also attenuate nicotine self-administration as well as alcohol self-administration in alcohol preferring rats^{36, 37}. Positive allosteric modulators of α4β2 nAChRs attenuate nicotine taking and seeking^{38, 39}. The classical non-selective nAChR antagonist mecamylamine has also shown efficacy in blocking nicotine self-administration¹⁴ and some efficacy in clinical trials when combined with a nicotine patch^{49, 41}. The selective α4β2 nAChR antagonist DHβE has also been demonstrated to attenuate nicotine self-administration in rats¹⁴. However, this compound, although very selective, has only moderate affinity and therefore rather poor potency for inhibition of the receptor in vitro⁴². Over the past several years, Carroll and colleagues have identified a number of epibatidine analogs with very high affinity and selective α4β2 nAChR agonist and antagonist activity⁴³⁻⁴⁸. One antagonist in particular, 4-nitro-PFEB, has near picomolar binding affinity to the α4β2 nAChR and inhibits receptor activity in vitro in the nanomolar range⁴⁶, although more recent studies have suggested partial agonist activity for this compound as well⁴⁷. 4-nitro-PFEB has also been demonstrated to block nicotine self-administration in rats and nicotine conditioned place preference in mice¹³

5 appears to have properties different than the compounds discussed above. 5 was not developed based upon known nicotinic structures, but rather by traditional medicinal chemistry starting, a priori, from very large, small molecule, mixture libraries²¹. Utilizing the hits that were identified in the original screening campaign as starting points a set of truncated analogs were synthesized and screened in order to identify the critical structural features driving potency and selectivity. This led to the monocyclic guanidine hits. A further medicinal chemistry effort was then undertaken to assess the potency and selectivity changes observed from a range of different R1 and R2 functionalities around the core monocyclic guanidine. 5 and its analog 13 have high affinity and selectivity in binding to the α4β2 nAChR as compared to α4β4, α3β2 and α3β4α5 nAChR. Affinity and selectivity are considerably higher than the “prototypical” α4β2 nAChR antagonist DHβE, though not as high as the epibatidine analogs. These compounds have no apparent agonist activity in cells transfected with α4β2 nAChR and potently inhibit receptor activity in vitro at concentration equivalent to their binding affinities. They have moderate activity at α3β4 nAChR at very high concentrations and no apparent effect on α7 nAChR. Lack of agonist activity in vitro is consistent with the inability of 5 to induce hypothermia.

When used in a preclinical model to determine potential efficacy as a smoking cessation medication, 5 potently (1 mg/kg) attenuated nicotine self-administration. However, the duration of action was short, reducing nicotine self-administration only in the first 30 min of the self-administration session. This is consistent with the time course of blood and brain concentration after systemic administration. However, 5 was more effective in reducing nicotine seeking in a model of relapse, completely inhibiting both nicotine prime-induced and cue-induced reinstatement of nicotine seeking.

In conclusion, we have identified a novel high affinity and selective α4β2 nAChR antagonist (5) that was originally derived from a small molecule mixture combinatorial library. 5 (and analog 13) have low nanomolar affinity for α4β2 nAChR. In vitro these compounds are devoid of agonist activity in cells transfected with rat α4β2 nAChR, act as antagonists of epibatidine at nanomolar concentrations, and have very weak partial agonist activity at α3β4 nAChR. In vivo 5 is also devoid of agonist activity and potently inhibits nicotine self-administration, as well as reinstatement of nicotine seeking. These results demonstrate that compounds like this could both reduce smoking and block relapse, suggesting that 5, with appropriate formulation to increase duration of action, has potential as a smoking cessation medication.

### Material and Methods

### Chemistry

The compounds were synthesized as described in the Scheme 1. The synthetic approach utilized was modified from a previously reported approach for obtaining cyclic guanidines from resin bound polyamines^{21, 48}. The solid phase synthesis was performed using the “tea-bag” methodology⁴⁹. Initially, 100 mg of p-methylbenzdrylamine (MBHA) resin (1.1 mmol/g, 100-200 mesh) was sealed in a mesh “tea-bag,” neutralized with 5% diisopropylethylamine (DIEA) in dichloromethane (DCM) and subsequently swelled with additional DCM washes. A Boc-amino acid (6 equiv) was coupled in dimethylformamide (0.1 M DMF) for 120 min in the presence of diisopropylcarbodiimide (DIC, 6 equiv) and 1-hydroxybenzotriazole hydrate (HOBt, 6 equiv) (1, Scheme 1). The Boc protecting group was removed with 55% Trifluoroacetic acid (TFA)/DCM for 30 min and subsequently neutralized with 5% DIEA/DCM (3×). Carboxylic acids were coupled using (6 equiv) in the presence of DIC (10 equiv) and HOBt (10 equiv) in DMF (0.1M) for 120 min (2, Scheme 1). All coupling reactions were monitored for completion by the ninhydrin test. The reduction was performed in a 4000 mL Wilmad LabGlass vessel under nitrogen. Borane in 1.0 M tetrahydrofuran complex solution was used in 40-fold excess for each amide bond. The vessel was heated to 65° C. and maintained at temperature for 72 h. The solution was then discarded, and the bags were washed with THF and methanol. Once completely dry, the bags were treated overnight with piperidine at 65° C. and washed several times with methanol, DMF, and DCM (3, Scheme 1). As previously reported, the reduction of polyamides with borane is free of racemization^{50, 51}. Before proceeding, completion of reduction was monitored by a control cleavage and analyzed by LCMS. Guanidine cyclization (E, Scheme 1) was performed with a 5-fold excess of cyanogen bromide (CNBr) in a 0.1 M anhydrous DCM solution. Following the cyclization, the bags were rinsed with DMF and DCM. The resin was cleaved with HF in the presence of anisole in an ice bath at 0° C. for 90 min (5, Scheme 1). The products were extracted using 95% acetic acid. Samples were then repeatedly frozen and lyophilized in 50% acetonitrile and water. Confirmation of the desired product was obtained by reverse phase LC-MS analysis using a Shimadzu 2010 LCMS system, consisting of a LC-20AD binary solvent pump, a DGU-20A degasser unit, a CTO-20A column oven, SIL-20A HT auto sampler, and SPD-M20A diode array set to scan 190-600 nm. Separation was achieved using a Phenomenex Luna C18 column (5 μm, 50 mm×4.6 mm i.d.) protecting with a Phenomenex C18 column guard (5 μm, 4×3.0 mm i.d.).

The crude product was purified using reverse phase mode on a Shimadzu Prominence preparative HPLC system consisting of LC-8A binary solvent pumps, a SCL-10A system controller, a SIL-10AP auto sampler, a FRC-10A fraction collector, and a Shimadzu SPD-20A UV detector. The wavelength was set at 214 nm during analysis. Chromatographic separations were obtained using a Phenomenex Luna C18 preparative column (5 μm, 150 mm×21.5 mm i.d.). The column was protected by a Phenomenex C18 column guard (5 μm, 15 mm×21.2 mm i.d.). Prominence prep software was used to set all detection and collection parameters. The mobile phases for HPLC purification were HPLC grade obtained from Sigma-Aldrich and Fisher Scientific. The mobile phase A consisted of water with 0.1% TFA and mobile phase B consisted of acetonitrile with 0.1% trifluoroacetic acid. Initial setting was set to 2% Mobile phase B and was gradually increased over time to achieve ideal separation for each compound. The peak corresponding to calculated m/z of the desired product was collected and concentrated. The concentrated pure product was analyzed on LC-MS and determined to be ≥95% purity based on peak area.

H1 NMR was obtained by dissolving approximately 5 mg of the pure material in deuterated DMSO. 1H NMR spectra were obtained utilizing the Bruker 400 Ascend (400 MHz). NMR chemical shifts were reported in δ (ppm) using the δ 2.50 signal of DMSO-d6, chloroform, or deuterium oxide (1H NMR) as an internal standard.

The following are embodiments of methods of making compounds of the present disclosure. In an aspect, solvents, acid, and other reactants can be changed to those known in the art to accomplish the reaction shown in the reactions schemes and consistent with the solvents, acids, and other reactants provided herein.

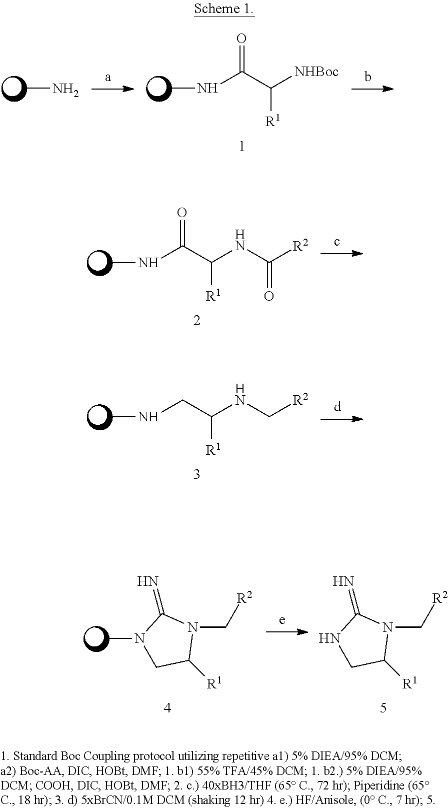

Synthetic schemes are shown below for the potential syntheses of the compounds from an aminoamide (possibly Resin bound) or a diamine (Resin bound or suitably protected). Reaction of the amine with a suitable electrophile followed by reduction (if necessary) provides the desired diamino compound. Cyclization of the diamine then provides the guanidine compound. “n” can be 0 to 10 or 0 to 5.

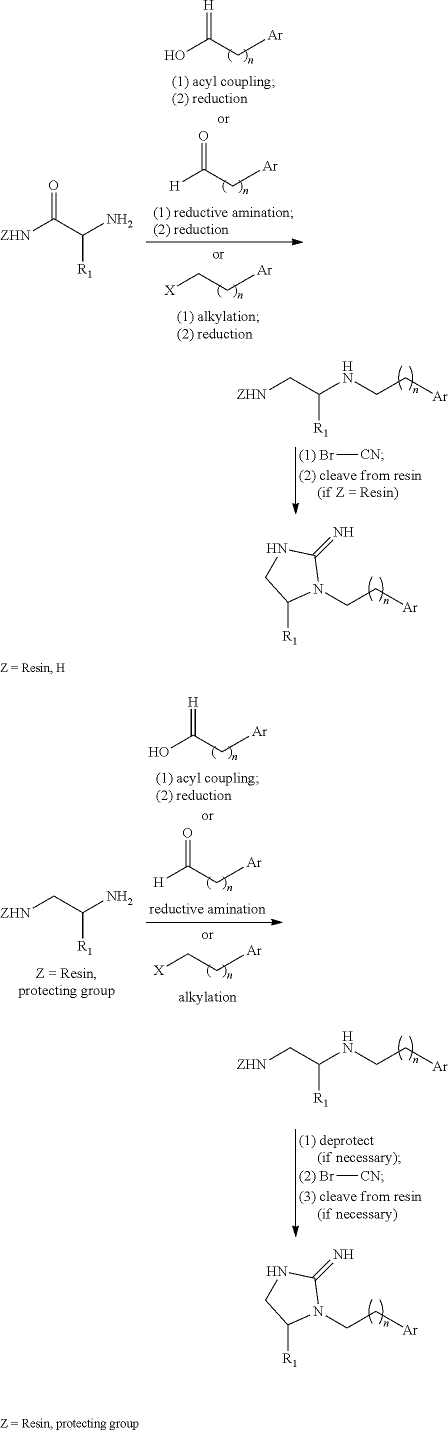

1-(3,4-Dichlorophenethyl)imidazolidin-2-imine (1) was synthesized with Boc-Glycine for the R1 reagent used in step a and 3,4-Dichlorophenylacetic acid used for the R2 reagent in step b. LC-MS (ESI+) m/z calculated for C₁₁H₁₃Cl₂N₃[258.05]+: MW found 257.9 and 259.9. 1H NMR (400 MHz, CHLOROFORM-d) δ ppm 2.82-2.97 (m, 2H) 3.42-3.67 (m, 5H) 7.02-7.21 (m, 1H) 7.28-7.49 (m, 2H) 8.52-8.79 (m, 1H) 8.98 (br. s., 1H) 10.06 (br. s., 1H).

1-(Pyridin-4-ylmethyl)imidazolidin-2-imine (2) was synthesized with Boc-L-Glycine for the R1 reagent used in step a and isonicotinic acid used for the R2 reagent in step b. LC-MS (ESI+) m/z calculated for C₉H₁₂N₄[177.11]+: MW found 176.9. 1H NMR (400 MHz, CHLOROFORM-d) δ ppm 2.67-2.94 (m, 1H) 3.46-3.72 (m, 4H) 4.44-4.64 (m, 2H) 8.22-8.50 (m, 1H) 8.51-8.64 (m, 2H).

1-Phenethylimidazolidin-2-imine(3) was synthesized with Boc-Glycine for the R1 reagent used in step a and phenylacetic acid used for the R2 reagent in step b. LC-MS (ESI+) m/z calculated for 190.13[190.13]+: MW found 190.0. 1H NMR (400 MHz, CHLOROFORM-d) δ ppm 2.14-2.41 (m, 2H) 2.93 (t, J=6.90 Hz, 2H) 3.31-3.53 (m, 2H) 3.53-3.75 (m, 3H) 7.25 (d, J=7.15 Hz, 2H) 7.28-7.45 (m, 3H).

(S)-5-Benzyl-1-(2-(pyridin-3-yl)ethyl)imidazolidin-2-imine (4) was synthesized with Boc-L-Phenylalanine for the R1 reagent used in step a and 3-pyridinyl acetic acid for the R2 reagent used in step b. LC-MS (ESI+) m/z calculated for C₁₇H₂ON₄[281.17]+: MW found 280.9. 1H NMR (400 MHz, DMSO-d₆) δ ppm 2.71-2.97 (m, 2H) 3.05-3.27 (m, 3H) 3.41-3.55 (m, 3H) 3.72 (ddd, J=15.12, 8.78, 6.84 Hz, 1H) 4.15-4.25 (m, 1H) 7.20-7.37 (m, 5H) 7.66-7.74 (m, 1H) 8.23-8.48 (m, 2H) 8.51 (s, 1H).

(S)-5-Methyl-1-(2-(pyridin-3-yl)ethyl)imidazolidin-2-imine (5) was synthesized with Boc-L-Alanine for the R1 reagent used in step a and 3-pyridinyl acetic acid for the R2 reagent used in step b. LC-MS (ESI+) m/z calculated for C₁₇H₁6N₄ [205.14]+: MW found 204.9. 1H NMR (400 MHz, CHLOROFORM-d₆) δ ppm 1.19-1.39 (m, 2H) 2.85-3.10 (m, 2H) 3.18-3.28 (m, 1H) 3.32-3.49 (m, 1H) 3.73 (t, J=9.29 Hz, 1H) 3.78-4.00 (m, 2H) 7.28-7.38 (m, 1H) 7.73 (d, J=7.78 Hz, 1H) 8.33-8.60 (m, 3H) 9.09 (br. s., 1H).

(S)-5-Isobutyl-1-(2-(pyridin-3-yl)ethyl)imidazolidin-2-imine (6) was synthesized with Boc-L-Leucine Isoleucine for the R1 reagent used in step a and 3-pyridinyl acetic acid for the R2 reagent used in step b. LC-MS (ESI+) m/z calculated for C₁₄H₂₂Nₐ [247.18]+: MW found 247.0. 1H NMR (400 MHz, CHLOROFORM-d₆) δ ppm 0.80-1.00 (m, 6H) 1.26-1.42 (m, 1H) 1.43-1.60 (m, 2H) 2.78-3.05 (m, 3H) 3.09-3.29 (m, 1H) 3.32-3.50 (m, 1H) 3.59-3.74 (m, 2H) 3.74-3.89 (m, 1H) 7.28-7.34 (m, 1H) 7.66 (d, J=7.78 Hz, 1H) 8.51 (br. s., 2H) 8.58 (d, J=14.56 Hz, 1H).

(S)-5-((R)-sec-Butyl)-1-(2-(pyridin-3-yl)ethyl)imidazolidin-2-imine (7) was synthesized with Boc-L-Isoleucine for the R1 reagent used in step a and 3-pyridinyl acetic acid for the R2 reagent used in step b. LC-MS (ESI+) m/z calculated for C₁₄H₂₂N₄[247.18]+: MW found 247.0. 1H NMR (400 MHz, CHLOROFORM-d₆) δ ppm 0.76-0.98 (m, 5H) 1.08-1.28 (m, 2H) 1.71-1.86 (m, 1H) 2.85-3.12 (m, 3H) 3.14-3.40 (m, 2H) 3.42-3.60 (m, 1H) 3.78 (ddd, J=9.98, 6.90, 3.45 Hz, 1H) 4.03 (dt, J=15.06, 7.40 Hz, 1H) 7.28-7.35 (m, 1H) 7.76 (d, J=7.78 Hz, 1H) 8.52 (d, J=9.79 Hz, 2H) 8.66 (br. s., 1H) 9.31 (br. s., 1H). 1-(2-(Pyridin-3-yl)ethyl)imidazolidin-2-imine (8) was synthesized with Boc-Glycine for the R1 reagent used in step a and 3-pyridinyl acetic acid for the R2 reagent used in step b. LC-MS (ESI+) m/z calculated for C₁₀H₁₄N₄[191.12]+: MW found 190.9. 1H NMR (400 MHz, DMSO-d₆) δ ppm 2.87 (t, J=7.47 Hz, 1H) 3.29-3.56 (m, 10H) 3.58-3.64 (m, 1H) 8.39-8.49 (m, 1H).

(S)-5-Isopropyl-1-(2-(pyridin-3-yl)ethyl)imidazolidin-2-imine (9) was synthesized with Boc-L-Valine for the R1 reagent used in step a and 3-pyridinyl acetic acid for the R2 reagent used in step b. LC-MS (ESI+) m/z calculated for C₁₃H₂ON₄[233.17]+: MW found 233.0. 1H NMR (400 MHz, DMSO-d₆) δ ppm 0.70-0.91 (m, 5H) 2.16 (dtd, J=13.63, 6.77, 6.77, 3.83 Hz, 1H) 2.78 (dq, J=9.07, 6.81 Hz, 1H) 2.92 (ddd, J=13.74, 8.97, 5.14 Hz, 1H) 3.17-3.39 (m, 4H) 3.39-3.55 (m, 1H) 3.58-3.76 (m, 1H) 3.92 (ddd, J=10.07, 6.43, 3.70 Hz, 1H) 7.14-7.36 (m, 1H) 7.77 (dt, J=7.87, 1.90 Hz, 1H) 8.21-8.46 (m, 2H) 8.55 (d, J=1.88 Hz, 1H).

(S)-1-(3-(2-Imino-3-(2-(pyridin-3-yl)ethyl)imidazolidin-4-yl)propyl)guanidine (10) was synthesized with Boc-L-Arginine Isoleucine for the R1 reagent used in step a and 3-pyridinyl acetic acid for the R2 reagent used in step b. LC-MS (ESI+) m/z calculated for C₁₄H₂₃N₇ [290.20]+: MW found 289.9 and 145.5. 1H NMR (400 MHz, DMSO-d6) δ ppm 1.42 (br. s., 1H) 1.50 (br. s., 1H) 2.78 (br. s., 1H) 3.08 (br. s., 1H) 3.14-3.33 (m, 2H) 3.39 (br. s., 3H)_{3.60} (br. s., 2H) 7.34 (br. s., 1H) 7.77 (br. s., 2H) 8.42 (br. s., 1H) 8.54 (br. s., 1H) 9.01 (br. s., 1H).

(S)-4-((2-Imino-3-(2-(pyridin-3-yl)ethyl)imidazolidin-4-yl)methyl)phenol (11) was synthesized with Boc-L-Tyrosine for the R1 reagent used in step a and 3-pyridinyl acetic acid for the R2 reagent used in step b. LC-MS (ESI+) m/z calculated for C₁₇H₂₀N₄O [297.16]+: MW found 296.9 and 149.0. 1H NMR (400 MHz, DMSO-d₆) δ ppm 2.53-2.68 (m, 1H) 2.75-3.02 (m, 2H) 3.09-3.25 (m, 1H) 3.38-3.54 (m, 3H) 3.71 (ddd, J=15.09, 8.75, 6.65 Hz, 1H) 4.06-4.14 (m, 1H) 6.48-6.73 (m, 2H) 6.92-7.07 (m, 2H) 7.28-7.36 (m, 1H) 7.73 (dt, J=7.84, 1.91 Hz, 1H) 8.20-8.46 (m, 2H)_{8.50}-8.53 (m, 1H).

(S)-5-(2-(Methylthio)ethyl)-1-(2-(pyridin-3-yl)ethyl)imidazolidin-2-imine (12) was synthesized with Boc-L-Methionine for the R1 reagent used in step a and 3-pyridinyl acetic acid for the R2 reagent used in step b. LC-MS (ESI+) m/z calculated for C₁₃H₂ON₄S[265.14]+: MW found 264.9. 1H NMR (400 MHz, DMSO-d₆) δ ppm 1.62-1.86 (m, 1H) 1.89-2.13 (m, 3H) 2.45 (t, J=7.65 Hz, 1H) 2.79 (dd, J=8.72, 6.84 Hz, 1H) 2.86-3.07 (m, 1H) 3.20-3.47 (m, 6H) 3.50-3.74 (m, 2H) 3.90-4.01 (m, 1H) 7.35 (ddd, J=7.81, 4.80, 0.69 Hz, 1H) 7.76 (dt, J=7.87, 1.90 Hz, 1H) 8.21-8.47 (m, 2H).

(R)-(2-Imino-3-(2-(pyridin-3-yl)ethyl)imidazolidin-4-yl)methanol (13) was synthesized with Boc-L-Serine for the R1 reagent used in step a and 3-pyridinyl acetic acid for the R2 reagent used in step b (Scheme 1). LC-MS (ESI+) m/z calculated for C₁₁H₁₆N₄O [M+H]+:221.1 MW found 220.9. 1H NMR (400 MHz, DEUTERIUM OXIDE) δ ppm 2.96 (br. s., 2H) 3.41 (br. s., 2H) 3.49-3.68 (m, 4H) 3.76 (br. s., 2H) 3.95 (br. s., 1H) 8.40 (br. s., 2H).

(S)-5-Methyl-1-(pyridin-3-ylmethyl)imidazolidin-2-imine (14) was synthesized with Boc-L-alanine for the R1 reagent used in step a and nicotinic acid used for the R2 reagent in step b. LC-MS (ESI+) m/z calculated for C₁₀H₁₄N₄[191.12]+: MW found 190.9. 1H NMR (400 MHz, DMSO-d6) δ ppm 3.35 (br. s., 8H) 3.53 (br. s., 3H) 8.54-8.81 (m, 1H).

1-(Pyridin-3-ylmethyl)imidazolidin-2-imine (15) was synthesized with Boc-Glycine for the R1 reagent used in step a and nicotinic acid used for the R2 reagent in step b. LC-MS (ESI+) m/z calculated for C₉H₁₂N₄[177.11]+: MW found 176.9. 1H NMR (400 MHz, DMSO-d₆) δ ppm 1.02-1.26 (m, 3H) 2.93-3.20 (m, 2H) 3.58-3.75 (m, 2H) 8.46-8.69 (m, 2H) 8.75-9.04 (m, 1H).

(S)-5-Methyl-1-phenethylimidazolidin-2-imine (16) was synthesized with Boc-L-alanine for the R1 reagent used in step a and phenylacetic acid used for the R2 reagent in step b. LC-MS (ESI+) m/z calculated for C₁₁H₁₆N₄[205.14]+: MW found 204.0.

(R)-(3-(3,4-Dichlorophenethyl)-2-iminoimidazolidin-4-yl)methanol (17) was synthesized with Boc-L-Serine Serine for the R1 reagent used in step a and 3,4-Dichlorophenylacetic acid used for the R2 reagent in step b. LC-MS (ESI+) m/z calculated for C₁₂H₁₆Cl₂N₃O[288.06]+: MW found 287.8 and 289.9. 1H NMR (400 MHz, DEUTERIUM OXIDE) d ppm 1.73-1.91 (m, 1H) 2.80-3.00 (m, 2H) 3.40-3.68 (m, 5H) 3.74-3.84 (m, 1H) 3.84-4.05 (m, 1H) 7.17 (dd, J=8.22, 2.07 Hz, 1H) 7.37-7.57 (m, 2H).

1-(3-Fluorophenethyl)imidazolidin-2-imine (18) was synthesized with Boc-Glycine for the R1 reagent used in step a and 3-Fluorophenylacetic acid for the R2 reagent used in step b. LC-MS (ESI+) m/z calculated for [208.12]+: MW found 207.0. 1H NMR (400 MHz, DEUTERIUM OXIDE) δ ppm 2.83-3.07 (m, 2H) 3.45-3.69 (m, 5H) 4.80 (br. s., 1H) 6.94-7.18 (m, 3H) 7.33 (q, J=7.40 Hz, 1H) 8.42 (br. s., 1H).

(S)-1-(3-Fluorophenethyl)-5-methylimidazolidin-2-imine (19) with Boc-L-alanine for the R1 reagent used in step a and 3-fluorophenylacetic acid used for the R2 reagent in step b. LC-MS (ESI+) m/z calculated for C₁₂H₁₆FN₃[222.13]+: MW found 222.0. 1H NMR (400 MHz, DEUTERIUM OXIDE) d ppm 1.12-1.28 (m, 3H) 2.81-2.95 (m, 2H) 3.13 (dd, J=9.54, 7.03 Hz, 1H) 3.45-3.67 (m, 3H) 3.80-3.99 (m, 1H) 6.97-7.14 (m, 3H) 7.33 (td, J=7.81, 6.46 Hz, 1H) 8.41 (br. s., 1H).

(R)-(3-(3-Fluorophenethyl)-2-iminoimidazolidin-4-yl)methanol (20) was synthesized with Boc-L-Serine for the R1 reagent used in step a and 3-Fluorophenylacetic acid used for the R2 reagent in step b. LC-MS (ESI+) m/z calculated for [238.13]+: MW found 238.0. 1H NMR (400 MHz, DEUTERIUM OXIDE) d ppm 2.85-3.02 (m, 2H) 3.42 (dd, J=9.91, 5.90 Hz, 1H) 3.51-3.69 (m, 4H) 3.75-3.93 (m, 2H) 6.98-7.15 (m, 3H) 7.26-7.41 (m, 1H) 8.41 (br. s., 1H).

1-(3-Bromophenethyl)imidazolidin-2-imine (211) was synthesized with Boc-Glycine for the R1 reagent used in step a and 3-bromophenylacetic acid used for the R2 reagent in step b. LC-MS (ESI+) m/z calculated for C₁₁H₁₄BrN₃[268.04]+: MW found 267.9 and 269.9 1H NMR (400 MHz, CHLOROFORM-d) δ ppm 1.75-1.94 (m, 1H) 2.74-2.95 (m, 2H) 3.38-3.64 (m, 6H) 7.34-7.47 (m, 2H) 8.61 (br. s., 1H).

(S)-1-(3-Bromophenethyl)-5-methylimidazolidin-2-imine (22) was synthesized with Boc-L-alanine for the R1 reagent used in step a and 3-bromophenylacetic acid used for the R2 reagent in step b. LC-MS (ESI+) m/z calculated for C₁₂H₁₆BrN₃[282.05]+: MW found 281.8 and 283.9. 1H NMR (400 MHz, DEUTERIUM OXIDE) d ppm 1.09-1.24 (m, 3H) 2.77-2.92 (m, 2H) 3.13 (dd, J=9.35, 7.22 Hz, 1H) 3.43-3.67 (m, 3H) 3.80-3.97 (m, 1H) 7.18-7.29 (m, 2H) 7.38-7.52 (m, 2H) 8.41 (s, 1H).

(R)-(3-(3-bromophenethyl)-2-iminoimidazolidin-4-yl)methanol (23) was synthesized with Boc-L-Serine for the R1 reagent used in step a and 3-Bromophenylacetic acid used for the R2 reagent in step b. LC-MS (ESI+) m/z calculated for C₁₂1-1₁₆BrN₃O[298.05]+: MW found 297.8 and 299.7. 1H NMR (400 MHz, DEUTERIUM OXIDE) δ ppm 2.82-2.96 (m, 2H) 3.40-3.67 (m, 5H) 3.73-3.92 (m, 2H) 7.19-7.31 (m, 2H) 7.42-7.50 (m, 2H) 8.44 (br. s., 1H).

(S)-1-(2-(6-chloropyridin-3-yl)ethyl)-5-methylimidazolidin-2-imine (24) was synthesized with Boc-L-alanine for the R1 reagent used in step a and 2-(6-chloropyridin-3-yl)acetic acid used for the R2 reagent in step b. LC-MS (ESI+) m/z calculated for C₁₁H₁₅C1N₄[238.10]+: MW found 238.9. 1H NMR (400 MHz, DEUTERIUM OXIDE) δ ppm 1.10-1.34 (m, 3H) 2.83-3.01 (m, 2H) 3.15 (dd, J=9.66, 7.03 Hz, 1H) 3.47-3.73 (m, 3H) 3.96 (dquin, J=9.14, 6.50, 6.50, 6.50, 6.50 Hz, 1H) 4.80 (br. s., 1H) 7.41 (d, J=8.28 Hz, 1H) 7.73 (dd, J=8.22, 2.45 Hz, 1H) 8.19 (d, J=2.26 Hz, 1H).

1-(2-(6-Chloropyridin-3-yl)ethyl)imidazolidin-2-imine (25) was synthesized with Boc-L-Glycine Tyrosine for the R1 reagent used in step a and 2-(6-chloropyridin-3-yl) acetic acid for the R2 reagent used in step b. LC-MS (ESI+) m/z calculated for C₁₀H₁₃ClN₄[225.08]+: MW found 224.9. 1H NMR (400 MHz, DEUTERIUM OXIDE) δ ppm 2.86-3.10 (m, 2H) 3.48-3.72 (m, 5H) 4.65 (s, 1H) 4.80 (br. s., 1H) 7.42 (d, J=8.28 Hz, 1H) 7.67-7.82 (m, 1H) 8.15-8.32 (m, 1H).

(R)-(3-(2-(6-chloropyridin-3-yl)ethyl)-2-iminoimidazolidin-4-yl)methanol (26) was synthesized with Boc-L-Serine for the R1 reagent used in step a and 2-(6-chloropyridin-3-yl) acetic acid used for the R2 reagent in step b. LC-MS (ESI+) m/z calculated for C₁₁H₁₅ClN₄O[254.09]+: MW found 254.9. 1H NMR (400 MHz, DEUTERIUM OXIDE) δ ppm 1.94-2.07 (m, 1H) 2.88-3.02 (m, 1H) 3.44 (dd, J=10.04, 5.90 Hz, 1H) 3.51-3.73 (m, 3H)_{3.80} (dd, J=12.61, 3.33 Hz, 1H) 3.93-4.10 (m, 1H) 4.65 (s, 1H) 4.76-5.03 (m, 2H) 7.43 (d, J=8.28 Hz, 1H) 7.75 (dd, J=8.28, 2.51 Hz, 1H)_{8.21} (d, J=2.38 Hz, 1H).

### Cell Culture.

HEK cells, containing rat α3β4, α4β4, α4β2, and α3β2 nAChR (obtained from Drs. Kenneth Kellar and Yingxian Xiao, Georgetown University), and α3β4α5 (obtained from Dr. Jon Lindsrom) were cultured in Dulbecco's modified Eagle's medium (DMEM), supplemented with 10% fetal bovine serum (FBS), 0.5% penicillin/streptomycin, and 0.4 mg/ml of geneticin, and were maintained in an atmosphere of 7.5% CO2 in a humidified incubator at 37° C. For binding assays, cells were passaged on 150-mm dishes and harvested when confluent.

### Binding Assays.

Cells were harvested by scraping the plates with a rubber policeman, suspended in 50 mM Tris buffer pH 7.4, homogenized using a Polytron Homogenizer, and the centrifugation was repeated twice at 20,000×g (13,500 rpm) for 20 min. For binding, the cell membranes were incubated with the test compounds or mixtures in the presence of 0.3 nM of [3H]epibatidine. After 2 h of incubation, at room temperature, samples were filtered, using a Tomtec cell harvester, through glass fiber filters that had been presoaked in 0.05% polyethyleneimine. Filters were counted on a betaplate reader (Wallac). Nonspecific binding was determined by using 1 μM of AT-1001 or 0.1 μM of unlabeled epibatidine. IC₅₀ values were determined by using the program Graphpad/PRISM. Ki values were calculated using the Cheng Prusoff transformation: Ki=IC₅₀/(1+L/Kd) where, L is radioligand concentration and Kd is the binding affinity of the radioligand, as determined previously by saturation analysis.

nAChR Functional Assays.

nAChR functional activity was determined by measuring nAChR-induced membrane potential change, which can be directly read by Molecular Devices Membrane Potential Assay Kit (Blue Dye) (Molecular Devices,) using the FlexStation 3® microplate reader (Molecular Devices). The HEK cells transfected with α3β4 or α4β2 nAChR are seeded in a 96-well plate (4,000 cells per well) one day prior to the experiments. For agonist assays, after brief washing, the cells are loaded with 225 μl of HBSS assay buffer (Hank's Balanced Salt Solution with 20 mM of HEPES, pH7.4), containing the blue dye, and incubated at 37° C. After 30 minutes, 25 μl of the appropriate compounds are dispensed into the wells by the FlexStation and nAChR stimulation-mediated membrane potential change is recorded every 3s for 120s by reading 565 nM fluorescence excited at 530 nM wavelength. For the antagonist assay, the cells are loaded with 200 μl HBSS buffer containing the blue dye and incubated at 37° C. After 20 minutes, 25 μl of test compounds is added, and after another 10 minutes, 25 μl of epibatidine (or nicotine) is added by the FlexStation, to a final concentration of 100 nM, with fluorescence measured as described above. The change in fluorescence represents the maximum response, minus the minimum response for each well. Graphpad PRISM was used to determine the EC₅₀ and IC₅₀ values.

### Animals.

Male Sprague Dawley rats obtained from Charles River (Portage, Mich.) and weighing 200-225 g at their arrival were used in this study. Rats were housed in groups of two in a room with a reverse 12 h light/12 h dark cycle (lights off at 07:30 AM). All experiments were conducted during the dark phase of the cycle. Animals were acclimatized for 7 days with water and chow (Teklad Diets, Madison, Wis.) and handled for 3 times before the experiments were started. Throughout all operant procedures, rats were food restricted (received 20 g of chow daily) with water freely accessible.

### Drugs and Chemicals.

5 was dissolved in a vehicle of 0.9% saline and injected subcutaneously at doses of (0.0, 0.3, 1.0 mg/kg). The injection volume was 1 ml/kg. The conotoxin TP 2212-59 was dissolved in 0.9% saline and injected icy at the volume of 1μ/rat. (−)-Nicotine hydrogen tartrate salt and alcohol were purchased from Sigma (St. Louis, Mo.). Alcohol was diluted to a concentration of 10% v/v in water and made available orally. Nicotine solution for i.v. injection (30 micrograms/kg/0.1 ml infusion) was obtained by dissolving the salt in 0.9% saline and the pH adjusted to 7.0-7.4 with 3 M sodium hydroxide. Nicotine self-administration dose is reported as free base concentration.

### Apparatus.

The self-administration boxes consisted of operant conditioning chambers (Med Associates, Inc., St. Albans, Vt.) enclosed in lit, sound attenuating, ventilated environmental cubicles. Each chamber was equipped with two retractable levers located in the front panel, laterally to a food pellet magazine. A pellet dispenser was positioned behind the front panel of the boxes. Chambers were also equipped with auditory stimuli presented via a speaker and visual stimuli located above the levers (cue lights) and near the top of the chamber opposite the lever on the front panel (house light). Infusions occurred by means of syringe pumps (Med Associates, Inc., St. Albans, Vt.) and liquid swivels (Instech Solomon, Plymouth Meeting, Pa.), connected to plastic tubing protected by a flexible metal sheath for attachment to the external catheter terminus. In the case of self-administration of one reinforcer alone, an infusion pump was activated by responses on the right (active) lever, while responses on the left (inactive) lever were recorded but did not result in any programmed consequences. Activation of the pump resulted in a delivery of 0.1 ml of the fluid. During co-administration of intravenous nicotine and oral alcohol, configuration of chambers slightly changed. A drinking receptacle substituted the food pellet magazine located between the two levers. The chambers were equipped with two active levers and two infusions pumps, one that delivered iv nicotine (0.1 ml) and one that delivered alcohol (0.1 ml) into the drinking receptacle. Thus, appropriate responding on the right lever resulted in activation of the pump containing nicotine while responding on the left lever resulted in activation of the pump that released alcohol, which was connected to the drinking receptacle through a PE-160 tube. A microcomputer controlled the delivery of reinforcers, presentation of auditory and visual stimuli, and recording of the behavioral data.

Food Training and i.v. Catheterization.

One week after arrival, all rats (except for those used for co-administration experiments) were trained for three days to lever-press for 45 mg food pellets (Test Diet, 5-TUM, Richmond, Ind.) under a fixed ratio 1 (FR-1) schedule of reinforcement in 30-min sessions. Then, animals underwent i.v surgery that occurred under isoflurane anesthesia. Incisions were made to expose the right jugular vein. A catheter made from micro-renathane tubing (inner diameter=0.020 in., outside diameter=0.037 in.) was subcutaneously positioned between the vein and the back as described in (Cippitelli et al 2016). For the duration of the experiment, the catheters were flushed daily with 0.2 ml of heparinized saline solution containing enrofloxacin (0.7 mg/ml). The self-administration experiments began 1 week after recovery from surgery.

### Effect of 5 on Nicotine Self-Administration

Rats (n=7) were trained to self-administer nicotine under a FR-1 schedule of reinforcement for five 2-hour daily sessions and under FR-3 for additional 9 sessions. Every three active lever presses resulted in the delivery of one nicotine dose (0.03 mg/kg/0.1 ml infusion). Following each nicotine infusion, a 20-second time out (TO) period occurred, during which responses at the active lever did not lead to programmed consequences. This TO period was concurrent with illumination of a cue light located above the active lever to signal delivery of the positive reinforcement. Additionally, an intermittent tone (7 kHz, 70 dB) was sounded throughout the 2-hour nicotine sessions. The rats were trained to self-administer nicotine until a stable baseline of reinforcement was established.

A Latin square, within-subject design was used for the drug treatment. The rats were injected subcutaneously with the drug (0.0, 0.3 and 1.0 mg/kg) 10 min before the beginning of the session. The animals were subjected to all treatments in counterbalanced order at least at 48 hours intervals between the drug test days.

### Effect of 5 on Nicotine Priming-Induced Reinstatement of Nicotine Seeking.

Rats (n=7) were trained to self-administer nicotine at the dose of 0.03 mg/kg/inf. in daily 2-hour sessions under a FR-1 followed by FR-3 schedule of reinforcement as described above. Following each nicotine infusion (0.1 ml), a 20-second timeout (TO) period occurred, during which pressing the active lever did not lead to programmed consequences. The TO was accompanied by illumination of a cue light located above the active lever to signal delivery of the positive reinforcement, while an intermittent tone was sounded throughout the sessions. Following 14 sessions an extinction phase was conducted. During 1-hour extinction sessions, the lever presses were no longer associated with nicotine delivery while all cues were presented to allow for their concomitant extinction. An extinction criterion was established for each animal individually and was defined as stable extinction responses during three consecutive days before testing. All animals reached the extinction criterion within 12 sessions. The day after the last extinction session, the rats were subjected to the reinstatement test by the subcutaneous injection of nicotine at the dose of 0.15 mg/kg. To evaluate the effect of AP-202 on nicotine priming-induced reinstatement, rats were administered the drug (0.3 and 1.0 mg/kg) or its vehicle (0.0 mg/kg) in a counterbalanced order (Latin square) 10 min before nicotine injection that was in turn administered 10 min before the 1-h reinstatement session. A 3-day interval occurred between drug tests, during which the animals were subjected to extinction sessions. The nicotine dose, time of injection and experimental design have been previously described²⁶.

### Effect of 5 on Cue-Induced Reinstatement of Nicotine Seeking.

Rats (n=7) were trained to lever press for nicotine at the dose of 0.03 mg/kg in daily 2-hour sessions under a FR-1 schedule of reinforcement for 5 days and under FR-3 for additional 9 days. Concurrently with the lever pressing, a 20-s time-out period was in effect. A stimulus predictive of nicotine (orange odor) was also presented immediately after the animals were placed in the operant chambers and immediately before the onset of every conditioning session⁵⁷. Furthermore, an intermittent tone (7 kHz, 70 dB) was sounded throughout the 2-hour nicotine sessions. Nicotine-reinforced responding was then extinguished in daily 1-h sessions that continued for 10 days. In this phase, neither nicotine nor the tone, the house light or the orange cue were available. On the day following the last extinction session (day 1), a 1-h reinstatement session was carried out without any drug treatment. Tone, odor and cue-light, but not nicotine, were presented, and reinstatement response rates (i.e., responses on the previously nicotine-associated lever) were recorded. These response rates were used to assign animals to treatment groups balanced for response rates for the drug treatment experiment that followed. To assess the effects of AP-202, reinstatement experiments were then conducted every third day [on days 4, 7, 10]. In a Latin-square counterbalanced order that paralleled that used for the self-administration studies, animals were pretreated with AP-202 (0.0, 0.3, 1.0 mg/kg, s.c.) 10 min prior the onset of the reinstatement sessions. Responding on the inactive lever was also recorded throughout the experiment.

### Assessment of Body Temperature:

Baseline rectal temperature was measured just prior to injection of AP-202 (1.0 mg/kg, sc) or vehicle (0.9% saline). Ten minutes later, nicotine (0.5 mg/kg, sc) or vehicle (0.9% saline) was injected, and temperature was measured again after 15, 30, 60 and 120 minutes.

### REFERENCES

1. Services., U. S. D. o. H. a. H. The Health Consequences of Smoking—50 Years of Progress: A Report of the Surgeon General; Atlanta, 2014.
2. Xu, X.; Bishop, E.; Kennedy, S.; Simpson, S.; Pechacek, T. Annual Healthcare Spending Attributable to Cigarette Smoking: An Update. American Journal of Preventive Medicine 2014, 48.
3. Organization, W. H. WHO Report on the Global Tobacco Epidemic, 2011.; Geneva, 2011.
4. Jorenby, D. E.; Hays, J. T.; Rigotti, N. A.; Azoulay, S.; Watsky, E. J.; Williams, K. E.; Billing, C. B.; Gong, J.; Reeves, K. R. Efficacy of varenicline, an alpha4beta2 nicotinic acetylcholine receptor partial agonist, vs placebo or sustained-release bupropion for smoking cessation: a randomized controlled trial. JAMA 2006, 296, 56-63.
5. Reus, V. I.; Smith, B. J. Multimodal techniques for smoking cessation: a review of their efficacy and utilisation and clinical practice guidelines. Int J Clin Pract 2008, 62, 1753-68.
6. Rigotti, N. A. Clinical practice. Treatment of tobacco use and dependence. N Engl J Med 2002, 346, 506-12.
7. Moore, T. J.; Furberg, C. D.; Glenmullen, J.; Maltsberger, J. T.; Singh, S. Suicidal behavior and depression in smoking cessation treatments. PLoS One 2011, 6, e27016.
8. Picciotto, M. R.; Zoli, M.; Rimondini, R.; Lena, C.; Marubio, L. M.; Pich, E. M.; Fuxe, K.; Changeux, J. P. Acetylcholine receptors containing the beta2 subunit are involved in the reinforcing properties of nicotine. Nature 1998, 391, 173-7.
9. Stolerman, I. P.; Shoaib, M. The neurobiology of tobacco addiction. Trends Pharmacol Sci 1991, 12, 467-73.
10. Perry, D. C.; Mao, D.; Gold, A. B.; McIntosh, J. M.; Pezzullo, J. C.; Kellar, K. J. Chronic nicotine differentially regulates alpha6- and beta3-containing nicotinic cholinergic receptors in rat brain. J Pharmacol Exp Ther 2007, 322, 306-15.
11. Perry, D. C.; Xiao, Y.; Nguyen, H. N.; Musachio, J. L.; Davila-Garcia, M. I.; Kellar, K. J. Measuring nicotinic receptors with characteristics of alpha4beta2, alpha3beta2 and alpha3beta4 subtypes in rat tissues by autoradiography. J Neurochem 2002, 82, 468-81.
12. Epping-Jordan, M. P.; Picciotto, M. R.; Changeux, J. P.; Pich, E. M. Assessment of nicotinic acetylcholine receptor subunit contributions to nicotine self-administration in mutant mice. Psychopharmacology (Berl) 1999, 147, 25-6.
13. Tobey, K. M.; Walentiny, D. M.; Wiley, J. L.; Carroll, F. I.; Damaj, M. I.; Azar, M. R.; Koob, G. F.; George, O.; Harris, L. S.; Vann, R. E. Effects of the specific alpha4beta2 nAChR antagonist, 2-fluoro-3-(4-nitrophenyl) deschloroepibatidine, on nicotine reward-related behaviors in rats and mice. Psychopharmacology (Berl) 2012, 223, 159-68.
14. Watkins, S. S.; Epping-Jordan, M. P.; Koob, G. F.; Markou, A. Blockade of nicotine self-administration with nicotinic antagonists in rats. Pharmacol Biochem Behav 1999, 62, 743-51.
15. Fowler, C. D.; Lu, Q.; Johnson, P. M.; Marks, M. J.; Kenny, P. J. Habenular α5 nicotinic receptor subunit signalling controls nicotine intake. Nature 2011, 471, 597-601.
16. Frahm, S.; Slimak, M. A.; Ferrarese, L.; Santos-Torres, J.; Antolin-Fontes, B.; Auer, S.; Filkin, S.; Pons, S.; Fontaine, J. F.; Tsetlin, V.; Maskos, U.; Ibanez-Tallon, I. Aversion to nicotine is regulated by the balanced activity of beta4 and alpha5 nicotinic receptor subunits in the medial habenula. Neuron 2011, 70, 522-35.
17. Harrington, L.; Vinals, X.; Herrera-Solis, A.; Flores, A.; Morel, C.; Tolu, S.; Faure, P.; Maldonado, R.; Maskos, U.; Robledo, P. Role of beta4* Nicotinic Acetylcholine Receptors in the Habenulo-Interpeduncular Pathway in Nicotine Reinforcement in Mice. Neuropsychopharmacology 2016, 41, 1790-802.
18. Morel, C.; Fattore, L.; Pons, S.; Hay, Y. A.; Marti, F.; Lambolez, B.; De Biasi, M.; Lathrop, M.; Fratta, W.; Maskos, U.; Faure, P. Nicotine consumption is regulated by a human polymorphism in dopamine neurons. Mol Psychiatry 2014, 19, 930-6.
19. McKee, S. A.; Harrison, E. L.; O'Malley, S. S.; Krishnan-Sarin, S.; Shi, J.; Tetrault, J. M.; Picciotto, M. R.; Petrakis, I. L.; Estevez, N.; Balchunas, E. Varenicline reduces alcohol self-administration in heavy-drinking smokers. Biol Psychiatry 2009, 66, 185-90.
20. Rezvani, A. H.; Slade, S.; Wells, C.; Petro, A.; Lumeng, L.; Li, T. K.; Xiao, Y.; Brown, M. L.; Paige, M. A.; McDowell, B. E.; Rose, J. E.; Kellar, K. J.; Levin, E. D. Effects of sazetidine-A, a selective alpha4beta2 nicotinic acetylcholine receptor desensitizing agent on alcohol and nicotine self-administration in selectively bred alcohol-preferring (P) rats. Psychopharmacology (Berl) 2010, 211, 161-74.
21. Steensland, P.; Simms, J. A.; Holgate, J.; Richards, J. K.; Bartlett, S. E. Varenicline, an alpha4beta2 nicotinic acetylcholine receptor partial agonist, selectively decreases ethanol consumption and seeking. Proc Natl Aced Sci USA 2007, 104, 12518-23.
22. Tapper, A. R.; McKinney, S. L.; Nashmi, R.; Schwarz, J.; Deshpande, P.; Labarca, C.; Whiteaker, P.; Marks, M. J.; Collins, A. C.; Lester, H. A. Nicotine activation of alpha4* receptors: sufficient for reward, tolerance, and sensitization. Science 2004, 306, 1029-32.
23. Chatterjee, S.; Steensland, P.; Simms, J. A.; Holgate, J.; Coe, J. W.; Hurst, R. S.; Shaffer, C. L.; Lowe, J.; Rollema, H.; Bartlett, S. E. Partial agonists of the alpha3beta4* neuronal nicotinic acetylcholine receptor reduce ethanol consumption and seeking in rats. Neuropsychopharmacology 2011, 36, 603-15.
24. Hendrickson, L. M.; Zhao-Shea, R.; Tapper, A. R. Modulation of ethanol drinking-in-the-dark by mecamylamine and nicotinic acetylcholine receptor agonists in C57BL/6J mice. Psychopharmacology (Berl) 2009, 204, 563-72.
25. Le, A. D.; Corrigall, W. A.; Harding, J. W.; Juzytsch, W.; Li, T. K. Involvement of nicotinic receptors in alcohol self-administration. Alcohol Clin Exp Res 2000, 24, 155-63.
26. Houghten, R. A.; Pinilla, C.; Giulianotti, M. A.; Appel, J. R.; Dooley, C. T.; Nefzi, A.; Ostresh, J. M.; Yu, Y.; Maggiora, G. M.; Medina-Franco, J. L.; Brunner, D.; Schneider, J. Strategies for the use of mixture-based synthetic combinatorial libraries: scaffold ranking, direct testing in vivo, and enhanced deconvolution by computational methods. J Comb Chem 2008, 10, 3-19.
27. Lopez-Vallejo, F.; Giulianotti, M. A.; Houghten, R. A.; Medina-Franco, J. L. Expanding the medicinally relevant chemical space with compound libraries. Drug Discov Today 2012, 17, 718-26.
28. Wu, J.; Zhang, Y.; Maida, L. E.; Santos, R. G.; Welmaker, G. S.; LaVoi, T. M.; Nefzi, A.; Yu, Y.; Houghten, R. A.; Toll, L.; Giulianotti, M. A. Scaffold ranking and positional scanning utilized in the discovery of nAChR-selective compounds suitable for optimization studies. J Med Chem 2013, 56, 10103-17.
29. Chang, Y. P.; Banerjee, J.; Dowell, C.; Wu, J.; Gyanda, R.; Houghten, R. A.; Toll, L.; McIntosh, J. M.; Armishaw, C. J. Discovery of a potent and selective alpha3beta4 nicotinic acetylcholine receptor antagonist from an alpha-conotoxin synthetic combinatorial library. J Med Chem 2014, 57, 3511-21.
30. Pajouhesh, H.; Lenz, G. R. Medicinal chemical properties of successful central nervous system drugs. NeuroRx 2005, 2, 541-53.
31. Cippitelli, A.; Wu, J.; Gaiolini, K. A.; Mercatelli, D.; Schoch, J.; Gorman, M.; Ramirez, A.; Ciccocioppo, R.; Khroyan, T. V.; Yasuda, D.; Zaveri, N. T.; Pascual, C.; Xie, X. S.; Toll, L. AT-1001: a high-affinity alpha3beta4 nAChR ligand with novel nicotine-suppressive pharmacology. Br J Pharmacol 2015, 172, 1834-45.
32. Erwin, B. L.; Slaton, R. M. Varenicline in the treatment of alcohol use disorders. Ann Pharmacother 2014, 48, 1445-55.
33. Carroll, F. I.; Ware, R.; Brieaddy, L. E.; Navarro, H. A.; Damaj, M. I.; Martin, B. R. Synthesis, nicotinic acetylcholine receptor binding, and antinociceptive properties of 2′-fluoro-3′-(substituted phenyl)deschloroepibatidine analogues. Novel nicotinic antagonist. J Med Chem 2004, 47, 4588-94.
34. Mihalak, K. B.; Carroll, F. I.; Luetje, C. W. Varenicline is a partial agonist at alpha4beta2 and a full agonist at alpha7 neuronal nicotinic receptors. Mol Pharmacol 2006, 70, 801-5.
35. Rollema, H.; Chambers, L. K.; Coe, J. W.; Glowa, J.; Hurst, R. S.; Lebel, L. A.; Lu, Y.; Mansbach, R. S.; Mather, R. J.; Rovetti, C. C.; Sands, S. B.; Schaeffer, E.; Schulz, D. W.; Tingley, F. D., 3rd; Williams, K. E. Pharmacological profile of the alpha4beta2 nicotinic acetylcholine receptor partial agonist varenicline, an effective smoking cessation aid. Neuropharmacology 2007, 52, 985-94.
36. Rollema, H.; Coe, J. W.; Chambers, L. K.; Hurst, R. S.; Stahl, S. M.; Williams, K. E. Rationale, pharmacology and clinical efficacy of partial agonists of alpha4beta2 nACh receptors for smoking cessation. Trends Pharmacol Sci 2007, 28, 316-25.
37. Chatterjee, S.; Bartlett, S. E. Neuronal nicotinic acetylcholine receptors as pharmacotherapeutic targets for the treatment of alcohol use disorders. CNS Neurol Disord Drug Targets 2010, 9, 60-76.
38. Hendrickson, L. M.; Zhao-Shea, R.; Pang, X.; Gardner, P. D.; Tapper, A. R. Activation of alpha4* nAChRs is necessary and sufficient for varenicline-induced reduction of alcohol consumption. J Neurosci 2010, 30, 10169-76.
39. Ahmed, A. I.; Ali, A. N.; Kramers, C.; Harmark, L. V.; Burger, D. M.; Verhoeven, W. M. Neuropsychiatric adverse events of varenicline: a systematic review of published reports. J Clin Psychopharmacol 2013, 33, 55-62.
40. Baker, T. B.; Piper, M. E.; Stein, J. H.; Smith, S. S.; Bolt, D. M.; Fraser, D. L.; Fiore, M. C. Effects of Nicotine Patch vs Varenicline vs Combination Nicotine Replacement Therapy on Smoking Cessation at 26 Weeks: A Randomized Clinical Trial. JAMA 2016, 315, 371-9.
41. Rasmussen, T.; Swedberg, M. D. Reinforcing effects of nicotinic compounds: intravenous self-administration in drug-naive mice. Pharmacol Biochem Behav 1998, 60, 567-73.
42. Cahill, K.; Lindson-Hawley, N.; Thomas, K. H.; Fanshawe, T. R.; Lancaster, T. Nicotine receptor partial agonists for smoking cessation. Cochrane Database Syst Rev 2016, CD006103.
43. Levin, E. D.; Rezvani, A. H.; Xiao, Y.; Slade, S.; Cauley, M.; Wells, C.; Hampton, D.; Petro, A.; Rose, J. E.; Brown, M. L.; Paige, M. A.; McDowell, B. E.; Kellar, K. J. Sazetidine-A, a selective alpha4beta2 nicotinic receptor desensitizing agent and partial agonist, reduces nicotine self-administration in rats. J Pharmacol Exp Ther 2010, 332, 933-9.
44. Rose, J. E.; Behm, F. M.; Westman, E. C. Nicotine-mecamylamine treatment for smoking cessation: the role of pre-cessation therapy. Exp Clin Psychopharmacol 1998, 6, 331-43.
45. Rose, J. E.; Behm, F. M.; Westman, E. C.; Levin, E. D.; Stein, R. M.; Ripka, G. V. Mecamylamine combined with nicotine skin patch facilitates smoking cessation beyond nicotine patch treatment alone. Clin Pharmacol Ther 1994, 56, 86-99.
46. Fitch, R. W.; Xiao, Y.; Kellar, K. J.; Daly, J. W. Membrane potential fluorescence: a rapid and highly sensitive assay for nicotinic receptor channel function. Proc Natl Acad Sci USA 2003, 100, 4909-14.
47. Carroll, F. I.; Lee, J. R.; Navarro, H. A.; Brieaddy, L. E.; Abraham, P.; Damaj, M. I.; Martin, B. R. Synthesis, nicotinic acetylcholine receptor binding, and antinociceptive properties of 2-exo-2-(2′-substituted-3′-phenyl-5′-pyridinyl)-7-azabicyclo[2.2.1]heptanes. Novel nicotinic antagonist. J Med Chem 2001, 44, 4039-41.
48. Carroll, F. I.; Liang, F.; Navarro, H. A.; Brieaddy, L. E.; Abraham, P.; Damaj, M. I.; Martin, B. R. Synthesis, nicotinic acetylcholine receptor binding, and antinociceptive properties of 2-exo-2-(2′-substituted 5′-pyridinyl)-7-azabicyclo[2.2.1]heptanes. Epibatidine analogues. J Med Chem 2001, 44, 2229-37.
49. Ondachi, P.; Castro, A.; Luetje, C. W.; Damaj, M. I.; Mascarella, S. W.; Navarro, H. A.; Carroll, F. I. Synthesis and nicotinic acetylcholine receptor in vitro and in vivo pharmacological properties of 2′-fluoro-3′-(substituted phenyl)deschloroepibatidine analogues of 2′-fluoro-3′-(4-nitrophenyl)deschloroepibatidine. J Med Chem 2012, 55, 6512-22.
50. Abdrakhmanova, G. R.; Damaj, M. I.; Carroll, F. I.; Martin, B. R. 2-Fluoro-3-(4-nitro-phenyl)deschloroepibatidine is a novel potent competitive antagonist of human neuronal alpha4beta2 nAChRs. Mol Pharmacol 2006, 69, 1945-52.
51. Carroll, F. I.; Ma, W.; Deng, L.; Navarro, H. A.; Damaj, M. I.; Martin, B. R. Synthesis, nicotinic acetylcholine receptor binding, and antinociceptive properties of 3′-(substituted phenyl)epibatidine analogues. Nicotinic partial agonists. J Nat Prod 2010, 73, 306-12.
52. Cippitelli, A.; Brunori, G.; Gaiolini, K. A.; Zaveri, N. T.; Toll, L. Pharmacological stress is required for the anti-alcohol effect of the alpha3beta4* nAChR partial agonist AT-1001. Neuropharmacology 2015, 93C, 229-236.
53. Le, A. D.; Funk, D.; Lo, S.; Coen, K. Operant self-administration of alcohol and nicotine in a preclinical model of co-abuse. Psychopharmacology (Berl) 2014, 231, 4019-29.
54. Batel, P.; Pessione, F.; Maitre, C.; Rueff, B. Relationship between alcohol and tobacco dependencies among alcoholics who smoke. Addiction 1995, 90, 977-80.
55. DiFranza, J. R.; Guerrera, M. P. Alcoholism and smoking. J Stud Alcohol 1990, 51, 130-5.
56. Cippitelli, A.; Schoch, J.; Debevec, G.; Brunori, G.; Zaveri, N. T.; Toll, L. A key role for the N/OFQ-NOP receptor system in modulating nicotine taking in a model of nicotine and alcohol co-administration. Sci Rep 2016, 6, 26594.
57. Cippitelli, A.; Damadzic, R.; Hansson, A. C.; Singley, E.; Sommer, W. H.; Eskay, R.; Thorsell, A.; Heilig, M. Neuropeptide Y (NPY) suppresses yohimbine-induced reinstatement of alcohol seeking. Psychopharmacology (Berl) 2010, 208, 417-26.
58. Cippitelli, A.; Damadzic, R.; Singley, E.; Thorsell, A.; Ciccocioppo, R.; Eskay, R. L.; Heilig, M. Pharmacological blockade of corticotropin-releasing hormone receptor 1 (CRH1R) reduces voluntary consumption of high alcohol concentrations in non-dependent Wistar rats. Pharmacol Biochem Behav 2012, 100, 522-9.
59. Le, A. D.; Lo, S.; Harding, S.; Juzytsch, W.; Marinelli, P. W.; Funk, D. Coadministration of intravenous nicotine and oral alcohol in rats. Psychopharmacology (Berl) 2010, 208, 475-86.
60. Pellow, S.; File, S. E. Anxiolytic and anxiogenic drug effects on exploratory activity in an elevated plus-maze: a novel test of anxiety in the rat. Pharmacol Biochem Behav 1986, 24, 525-9.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

We claim:

1. A composition, comprising a α4β2 nAChR antagonist having a structure: 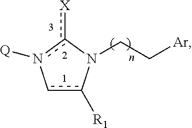
and stereoisomers thereof,
wherein X is independently selected from H, NH, NH₂, OH, an alkyl group, or a substituted alkyl group, wherein a double bond 3 is formed based on X;
wherein R₁ is independently selected from an alkyl group, a substituted alkyl group, hydroxyl alkyl group, or a substituted hydroxyl alkyl group;
wherein Q is H or is not present and when not present, a double bond 2 is formed;
wherein n is 0, 1, 2, 3, 4, or 5; and
wherein Ar is independently selected from an heteroaryl group.

2. The composition of claim 1, having the structure:

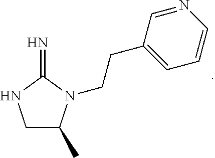

3. The composition of claim 1, having the structure:

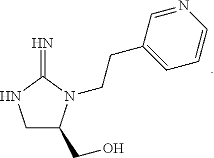 [SMILES: N=C1NC[C@H](CO)N1CCc1cccnc1]

4. The composition of claim 1, wherein the heteroaryl group is selected from: pyridyl, imidazoyl, pyridineimidazoyl, oxazolyl, or a halogen substituted pyridyl.

5. A pharmaceutical composition, comprising a therapeutically effective amount of a α4β2 nAChR antagonist or a pharmaceutically acceptable salt of the α4β2 nAChR antagonist, and a pharmaceutically acceptable carrier, to treat a condition, wherein the α4β2 nAChR antagonist has the following structure: 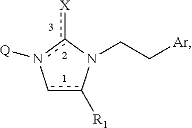
and stereoisomers thereof,
wherein X is independently selected from H, NH₂, OH, an alkyl group, or a substituted alkyl group, wherein a double bond 3 is formed based on X;
wherein R₁ is independently selected from an alkyl group, a substituted alkyl group, hydroxyl alkyl group, or a substituted hydroxyl alkyl group;
wherein Q is H or is not present and when not present, a double bond 2 is formed;
wherein n is 0, 1, 2, 3, 4, or 5; and
wherein Ar is independently selected from a heteroaryl group.

6. The pharmaceutical composition of claim 5, wherein the heteroaryl group is selected from: pyridyl, imidazoyl, pyridineimidazoyl, oxazolyl, or a halogen substituted pyridyl.

7. The pharmaceutical composition of claim 5, wherein the α4β2 nAChR antagonist is selected from one of the following structures:

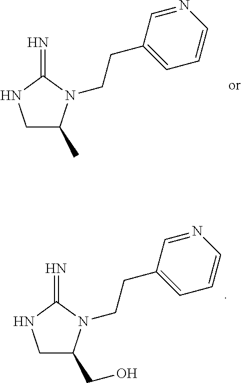 [SMILES: C[C@H]1CNC(=N)N1CCc1cccnc1 ; N=C1NC[C@H](CO)N1CCc1cccnc1]

8. A method of treating a condition comprising: delivering to a subject in need thereof, a pharmaceutical composition, wherein the pharmaceutical composition includes a therapeutically effective amount of a α4β2 nAChR antagonist or a pharmaceutically acceptable salt of the α4β2 nAChR antagonist, and a pharmaceutically acceptable carrier, to treat the condition, wherein the α4β2 nAChR antagonist has the following structure: 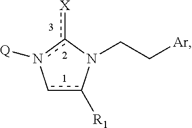
and stereoisomers thereof,
wherein X is independently selected from H, NH₂, OH, an alkyl group, or a substituted alkyl group, wherein a double bond 3 is formed based on X;
wherein R₁ is independently selected from an alkyl group, a substituted alkyl group, hydroxyl alkyl group, or a substituted hydroxyl alkyl group;
wherein Q is H or is not present and when not present, a double bond 2 is formed;
wherein n is 0, 1, 2, 3, 4, or 5; and
wherein Ar is independently selected from a heteroaryl group.

9. The method of claim 8, wherein the α4β2 nAChR antagonist is selected from one of the following structures:

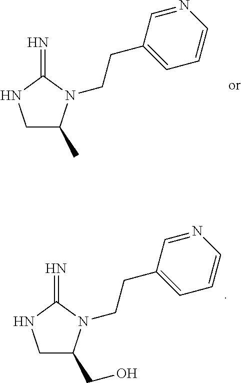 [SMILES: C[C@H]1CNC(=N)N1CCc1cccnc1 ; N=C1NC[C@H](CO)N1CCc1cccnc1]

10. The method of claim 8, wherein the heteroaryl group is selected from: pyridyl, imidazoyl, pyridineimidazoyl, oxazolyl, or a halogen substituted pyridyl.

11. The method of claim 8, wherein the pharmaceutical composition may comprise a plurality of α4β2 nAChR antagonists.

12. (canceled)

13. The method of claim 8, wherein the condition is nicotine addiction.

14. The method of claim 8, wherein the condition is relapse of nicotine addiction.

15. The method of claim 8 wherein the condition is obsessive-compulsive disorder.

16. The method of claim 8, wherein the condition is depression, anxiety, panic disorder.

17. The method of claim 8, wherein the condition is anxiety or generalized anxiety disorder.

18-26. (canceled)
